(19) United States
(12) Patent Application Publication
(10) Pub. No.: US 2023/0272340 A1
(43) Pub. Date: Aug. 31, 2023

(54) COMPOSITIONS AND METHODS FOR ANTIGEN IDENTIFICATION

(71) Applicant: RootPath Genomics, Inc., Watertown, MA (US)

(72) Inventors: Xi CHEN, Newton, MA (US); Ely PORTER, Medford, MA (US); Julia LEUNG, Medford, MA (US)

(21) Appl. No.: 18/019,174

(22) PCT Filed: Aug. 12, 2021
(86) PCT No.: PCT/US2021/045754

§ 371 Date: Feb. 1, 2023

Related U.S. Application Data
(60) Provisional application No. 63/065,074, filed on Aug. 13, 2020.
(60) Provisional application No. 63/119,066, filed on Nov. 30, 2020.

(51) Int. Cl. C12N 5/0783; C07K 14/725; C07K 14/74; C07K 16/24; C12Q 1/6869

(52) U.S. Cl. CPC C12N 5/0636; C07K 14/7051; C07K 14/70539; C07K 16/24; C12Q 1/6869; C07K 2319/00; C07K 2317/622

(57) ABSTRACT

The present disclosure provides compositions and methods for antigen identification. The methods provided herein comprise capturing polypeptides such as cytokines secreted by TCR-expressing cells (e.g., T cells) by antigen-presenting cells (APCs) presenting the antigens recognized by the TCRs of the TCR-expressing cells. The APCs can be detected or selected for antigen identification.

### CROSS-REFERENCE

This application claims the benefit of U.S. Provisional Patent Application No. 63/065,074, filed Aug. 13, 2020, and U.S. Provisional Patent Application No. 63/119,066, filed Nov. 30, 2020, each of which is incorporated herein by reference in its entirety.

### BACKGROUND OF THE INVENTION

The T-cell receptor (TCR) can be responsible for the recognition of the antigen-major histocompatibility complex, leading to the initiation of an inflammatory response. Many T cell subsets can exist, including cytotoxic T cells and helper T cells. Cytotoxic T cells (also known as CD8+ T cells) can kill abnormal cells, for example virus-infected or tumor cells. Helper T cells (also known as CD4+ T cells) can aid in the activation and maturation of other immune cells. Both cytotoxic and helper T cells can carry out their function subsequent to the recognition of specific target antigens which triggers their respective responses. The antigen specificity of a T cell can be defined by the TCR expressed on the surface of the T cell. T cell receptors can be heterodimer proteins composed of two polypeptide chains, most commonly an alpha and beta chain, but a minority of T cells can express a gamma and delta chain. The specific amino acid sequence of the TCR and the resultant three-dimensional structure can define the TCR antigen specificity and affinity. The amino acid and coding DNA sequences of the TCR chains for any individual T cell may be unique or at very low abundance in an organism's entire TCR repertoire, since there are a vast number of possible TCR sequences. This large sequence diversity is achieved during T cell development through a number of cellular mechanisms and may be a critical aspect of the immune system's ability to respond to a huge variety of potential antigens.

TCR target antigens are peptides which can be displayed by an antigen presenting cell (APC). On the cell surface of an APC, a peptide is presented to a T cell in complex with a major histocompatibility complex (MHC) protein, referred to as human leukocyte antigens (HLA) in humans. MHC class I proteins can display peptide antigens to cytotoxic T cells and MHC class II proteins can display peptide antigens to helper T cells. MHC genes can be highly polymorphic, and thus the specific set of MHC proteins for any organism may differ substantially from most or all others within the species.

### SUMMARY OF THE INVENTION

Recognized herein are needs for methods and compositions to screen and determine the antigen specificity of large numbers of TCRs in a high-throughput and cost effective manner. The compositions and methods provided herein can directly screen and select large numbers of APCs comprising antigens that can be recognized by the TCRs.

In an aspect, the present disclosure provides a method of identifying an antigen-presenting cell (APC) comprising an antigen recognized by a T-cell receptor (TCR), the method comprising: (a) providing a plurality of APCs, each comprising a different antigen complexed with a major histocompatibility complex (WIC) molecule, and a plurality of TCR-expressing cells comprising a TCR; (b) partitioning the plurality of APCs and the plurality of TCR-expressing cells into a plurality of compartments, a compartment of the plurality of compartments comprising (i) an APC of the plurality of APCs and (ii) at least one TCR-expressing cell of the plurality of TCR-expressing cells; (c) binding an antigen complexed with an WIC molecule of the APC to the TCR of the at least one TCR-expressing cell within the compartment, wherein the at least one TCR-expressing cell secretes a polypeptide upon binding, and wherein the polypeptide that is secreted binds to a catch agent associated with the APC; and (d) selecting the APC based on the polypeptide bound to the catch agent associated with the APC, thereby identifying the APC comprising the antigen recognized by the TCR.

In some embodiments, the polypeptide is endogenous or exogenous of the at least one TCR-expressing cell. In some embodiments, the polypeptide is engineered to be secreted by the at least one TCR-expressing cell. In some embodiments, the polypeptide is a cytokine. In some embodiments, the catch agent is associated with the APC indirectly. In some embodiments, the compartment of the plurality of compartments of (b) further comprises a solid support. In some embodiments, the solid support is associated with the APC. In some embodiments, the solid support is modified with an antibody or fragment thereof, which antibody or fragment thereof binds to a cell surface protein of the APC. In some embodiments, the catch agent is associated with the solid support. In some embodiments, the catch agent is associated with the solid support via a crosslinker or an affinity binding pair. In some embodiments, the solid support is a bead or a hydrogel particle.

In some embodiments, the catch agent is associated with the APC directly. In some embodiments, the catch agent is associated with the APC directly via covalent or non-covalent interaction. In some embodiments, the catch agent is associated with the APC via click chemistry. In some embodiments, the catch agent comprises a conjugation handle, and the APC comprises an additional conjugation handle that reacts with the conjugation handle via click chemistry. In some embodiments, the conjugation handle and the additional conjugation handle comprise TCO and tetrazine, azide and alkyne, or azide and DBCO. In some embodiments, the catch agent is associated with the APC via an affinity binding pair. In some embodiments, the affinity binding pair comprises streptavidin and biotin.

In some embodiments, the catch agent is a membrane-bound catch agent. In some embodiments, the membrane-bound catch agent is a transmembrane protein. In some embodiments, the transmembrane protein is a receptor expressed by the APC. In some embodiments, the receptor is exogenously expressed by the APC. In some embodiments, the catch agent is a soluble catch agent. In some embodiments, the soluble catch agent is (i) a molecule expressed and/or secreted by the APC or the TCR-expressing cell or (ii) a molecule supplied within the compartment. In some embodiments, the soluble catch agent has specificity to a cell surface protein of the APC and the polypeptide. In some embodiments, the soluble catch agent binds to both a cell surface protein of the APC and the polypeptide. In some embodiments, the cell surface protein is an endogenous protein or an exogenous protein of the APC. In some embodiments, the soluble catch agent is an antibody or a fragment thereof. In some embodiments, the antibody is a bispecific antibody (BsAb). In some embodiments, the BsAb is an antibody produced by a quadroma cell. In some embodiments, the BsAb is a heterodimeric antibody or a fragment thereof. In some embodiments, the BsAb is a recombinant protein or a bispecific fusion protein. In some embodiments, the BsAb comprises a first antigen binding domain targeting the cell surface protein of the APC and a second antigen binding domain targeting the polypeptide. In some embodiments, the first antigen binding domain and the second antigen binding domain are linked by a linker. In some embodiments, the linker is a chemical linker. In some embodiments, the first antigen binding domain or the second antigen binding domain is an IgG, a scFv or a sdAb. In some embodiments, the cell surface protein is CD11b, CD11c, CD14, CD80, CD86, B7-1, B7-2, CD18, CD29, CD31, CD40, CD43, CD44, CD45, CD54, CD58, CD83, CD86, IFN-γ receptor, IL-2 receptor, ICAM-1, Fcγ receptor, CMRF-44, CMRF-56, DCIR, or DECTIN-1. In some embodiments, the polypeptide is TNFα tumor necrosis factor alpha (TNFα), TNFβ, interleukin (IL)-1α, IL-1β, IL-2, IL-4, IL-5, IL-6, IL-9, IL-10, IL-12, IL-13, IL-15, IL-17, IL-18, IL-21, IL-22, IL-25, transforming growth factor beta (TGF-β), or interferon (IFN-γ). In some embodiments, the cell surface protein is CD45, and wherein the polypeptide is selected from the group consisting of IL-2, IL-4, IL-10, IL-12, IFN-γ and TNFβ. In some embodiments, the polypeptide is IL-2. In some embodiments, the cell surface protein is CD11b, and wherein the polypeptide is selected from the group consisting of IL-2, IL-4, IL-10, IL-12, IFN-γ, TNFα and TNFβ. In some embodiments, the polypeptide is IFN-γ. In some embodiments, selecting in (d) comprises contacting the polypeptide bound to the catch agent associated with the APC with a detection agent, wherein the detection agent binds to the polypeptide. In some embodiments, the detection agent is an antibody or fragment thereof. In some embodiments, the detection agent has specificity to the polypeptide. In some embodiments, the detection agent and the catch agent bind to different epitopes of the polypeptide. In some embodiments, the detection agent comprises a signal or an affinity tag. In some embodiments, the signal is a detectable label. In some embodiments, the detectable label is a fluorescent label.

In some embodiments, the method further comprise, subsequent to (c), releasing the plurality of APCs from the plurality of compartments, wherein the APC bound with the polypeptide is released from the compartment. In some embodiments, the plurality of compartments is a plurality of droplets, and wherein releasing comprises demulsifying the plurality of droplets. In some embodiments, selecting comprises selecting the APC comprising the antigen recognized by the TCR based on the signal or the affinity tag of the detection agent. In some embodiments, selecting comprises sorting the plurality of APCs by flow cytometry based on the signal from the detection agent, and wherein the APC comprising the antigen recognized by the TCR is selected. In some embodiments, selecting comprises capturing the affinity tag of the detection agent.

In some embodiments, the APC comprises a nucleic acid molecule encoding the antigen. In some embodiments, the method further comprises sequencing the nucleic acid or a derivative thereof to identify the antigen. In some embodiments, the plurality of APCs comprises artificial APCs (aAPCs). In some embodiments, the aAPCs comprises cells engineered to express an MHC molecule. In some embodiments, the cells engineered to express an WIC molecule is a cell line or cells isolated from a subject. In some embodiments, the cell line is K562. In some embodiments, the cells isolated from a subject are tumor cells. In some embodiments, the plurality of APCs comprises professional APCs or non-professional APCs. In some embodiments, the professional APCs comprise dendritic cells, macrophages, or B cells. In some embodiments, the non-professional APCs comprise fibroblasts, thymic epithelial cells, thyroid epithelial cells, glial cells, pancreatic beta cells, or vascular endothelial cells. In some embodiments, the plurality of TCR-expressing cells comprises T cells. In some embodiments, the T cells comprise a CD4+ or CD8+ T cell. In some embodiments, the T cells comprise a cytotoxic T cell, an ancillary T cell, a natural killer T cell, an alpha beta T cell, a gamma delta T cell, a regulatory T cell or a memory T cell. In some embodiments, the T cells are a cell line. In some embodiments, the compartment of the plurality of compartments comprise a single APC. In some embodiments, each compartment of the plurality of compartments comprises a single APC. In some embodiments, the plurality of compartments comprises at least about 20, 100, 1,000, or 10,000, 100,000 or more compartments. In some embodiments, the plurality of TCRs comprises at least about 2 times more cells than the plurality of APCs.

In another aspect, the present disclosure provides a method of identifying an antigen-presenting cell (APC) comprising an antigen recognized by a T-cell receptor (TCR), the method comprising: (a) providing a plurality of APCs, each comprising a different antigen complexed with a major histocompatibility complex (WIC) molecule, and a plurality of TCR-expressing cells comprising a TCR; (b) partitioning the plurality of APCs and the plurality of TCR-expressing cells into a plurality of compartments, a compartment of the plurality of compartments comprising (i) an APC of the plurality of APCs and (ii) at least one TCR-expressing cell of the plurality of TCR-expressing cells; (c) binding an antigen complexed with an WIC molecule of the APC to the TCR of the at least one TCR-expressing cell within the compartment, wherein the at least one TCR-expressing cell secretes a polypeptide upon binding, and wherein the polypeptide that is secreted binds to a catch agent associated with the APC; and (d) detecting the polypeptide bound to the catch agent associated with the APC, thereby identifying the APC comprising the antigen recognized by the TCR. In some embodiments, detecting in (d) comprises contacting the polypeptide bound to the catch agent associated with the APC with a detection agent, wherein the detection agent binds to the polypeptide.

In another aspect, the present disclosure provides a method of identifying an antigen-presenting cell (APC) comprising an antigen recognized by a T-cell receptor (TCR), comprising: (a) providing (i) a plurality of antigen-presenting cells (APCs) comprising a first APC and a second APC, wherein the first APC comprises a first antigen that is recognized by the TCR and the second APC comprises a second antigen that is not recognized by the TCR, and (ii) a plurality of TCR-expressing cells comprising the TCR; (b) contacting the first APC and the second APC with the plurality of TCR-expressing cells, wherein a TCR-expressing cell of the plurality binds to the first antigen of the first APC and secretes a polypeptide upon binding, wherein the polypeptide that is secreted binds to a first catch agent associated with the first APC, and wherein the polypeptide is diffusion-restricted such that it does not bind to a second catch agent associated with the second APC; and (c) detecting the polypeptide bound to the first catch agent associated with the first APC, thereby identifying the first APC as the APC comprising the antigen recognized by the TCR. In some embodiments, an amount of the polypeptide on the second APC is at least 5 times less than an amount of the polypeptide on the first APC. In some embodiments, the second APC is at least about 0.1 mm from the first APC.

In another aspect, the present disclosure provides a method of identifying an antigen-presenting cell (APC) comprising an antigen recognized by a T-cell receptor (TCR), comprising: (a) providing (i) a plurality of antigen-presenting cells (APCs) comprising a first APC and a second APC, wherein the first APC comprises a first antigen that is recognized by the TCR and the second APC comprises a second antigen that is not recognized by the TCR, and (ii) a plurality of TCR-expressing cells comprising the TCR; (b) contacting the first APC and the second APC with the plurality of TCR-expressing cells, wherein a TCR-expressing cell of the plurality binds to the first antigen of the first APC and secretes a polypeptide upon binding, wherein the polypeptide binds to a first catch agent associated with the first APC, and wherein an amount of the polypeptide on the second APC is at least 5 times less than an amount of the polypeptide on the first APC; and (c) detecting the polypeptide bound to the first catch agent associated with the first APC, thereby identifying the first APC as the APC comprising the antigen recognized by the TCR. In some embodiments, the polypeptide is diffusion-restricted. In some embodiments, the second APC is at least about 0.1 mm from the first APC.

In another aspect, the present disclosure provides a method of identifying an antigen-presenting cell (APC) comprising an antigen recognized by a T-cell receptor (TCR), comprising: (a) providing (i) a plurality of antigen-presenting cells (APCs) comprising a first APC and a second APC, wherein the first APC comprises a first antigen that is recognized by the TCR and the second APC comprises a second antigen that is not recognized by the TCR, and (ii) a plurality of TCR-expressing cells comprising the TCR; (b) contacting the first APC and the second APC with the plurality of TCR-expressing cells, wherein a TCR-expressing cell of the plurality binds to the first antigen of the first APC and secretes a polypeptide upon binding, wherein the polypeptide binds to a first catch agent associated with the first APC, and wherein the second APC is at least about 0.1 mm from the first APC; and (c) detecting the polypeptide bound to the first catch agent associated with the first APC, thereby identifying the first APC as the APC comprising the antigen recognized by the TCR. In some embodiments, an amount of the polypeptide on the second APC is at least 5 times less than an amount of the polypeptide on the first APC. In some embodiments, the polypeptide is diffusion-restricted.

In some embodiments, the first APC and the second APC are in the same compartment. In some embodiments, the first or the second catch agent has specificity to the polypeptide.

In some embodiments, the first or the second catch agent is associated with the APC indirectly. In some embodiments, the compartment of the plurality of compartments of (b) further comprises a solid support. In some embodiments, the solid support is associated with the APC. In some embodiments, the solid support is modified with an antibody or fragment thereof, which antibody or fragment thereof binds to a cell surface protein of the APC. In some embodiments, the first or the second catch agent is associated with the solid support. In some embodiments, the first or the second catch agent is associated with the solid support via a crosslinker or an affinity binding pair. In some embodiments, the solid support is a bead or a hydrogel particle. In some embodiments, the first or the second catch agent is associated with the APC directly. In some embodiments, the first or the second catch agent is associated with the APC directly via covalent or non-covalent interaction. In some embodiments, the first or the second catch agent is associated with the APC via click chemistry. In some embodiments, the first or the second catch agent comprises a conjugation handle, and the APC comprises an additional conjugation handle that reacts with the conjugation handle via click chemistry. In some embodiments, the conjugation handle and the additional conjugation handle comprise TCO and tetrazine, azide and alkyne, or azide and DBCO. In some embodiments, the first or the second catch agent is associated with the APC via an affinity binding pair. In some embodiments, the affinity binding pair comprises streptavidin and biotin.

In some embodiments, the first or the second catch agent is a membrane-bound catch agent. In some embodiments, the membrane-bound catch agent is a transmembrane protein. In some embodiments, the transmembrane protein is a receptor expressed by the first or the second APC. In some embodiments, the receptor is exogenously expressed. In some embodiments, the first or the second catch agent is a soluble catch agent. In some embodiments, the soluble catch agent is (i) a molecule expressed and/or secreted by the first or the second APC or the TCR-expressing cell or (ii) a molecule supplied within the same compartment. In some embodiments, the soluble catch agent has specificity to a cell surface protein of the first APC and the polypeptide. In some embodiments, the soluble catch agent binds to both a cell surface protein of the first APC and the polypeptide. In some embodiments, the cell surface protein is an endogenous protein or an exogenous protein of the first APC. In some embodiments, the soluble catch agent is an antibody or a fragment thereof. In some embodiments, the antibody is a bispecific antibody (BsAb). In some embodiments, detecting in (c) comprises contacting the polypeptide bound to the first catch agent associated with the APC with a detection agent, wherein the detection agent binds to the polypeptide. In some embodiments, the detection agent is an antibody or fragment thereof. In some embodiments, the detection agent has specificity to the polypeptide. In some embodiments, the detection agent comprises a signal. In some embodiments, the signal is a detectable label. In some embodiments, the detectable label is a fluorescent label. In some embodiments, the first APC comprises a nucleic acid molecule encoding the first antigen. In some embodiments, the method further comprises sequencing the nucleic acid molecule or a derivative thereof to identify the first antigen. In some embodiments, the plurality of TCRs comprises at least about 2 times more cells than the plurality of APCs.

In another aspect, the present disclosure provides a method for labeling an antigen-presenting cell (APC) comprising an antigen as being recognized by a T-cell receptor (TCR), comprising: contacting an APC comprising an antigen complexed with a major histocompatibility complex (WIC) molecule recognized by a TCR with a TCR-expressing cell comprising the TCR, wherein the TCR-expressing cell secretes a polypeptide upon binding the APC, wherein the polypeptide binds to a catch agent associated with the APC, thereby labeling the APC with the polypeptide, and wherein the catch agent is (i) a soluble catch agent having specificity to both a cell surface protein of the APC and the polypeptide, or (ii) an exogenously expressed protein having specificity to the polypeptide. In some embodiments, the exogenously expressed protein is a membrane-bound protein.

In another aspect, the present disclosure provides a method of identifying an additional TCR that recognizes an antigen identified by a method provided herein, comprising contacting a plurality of cells, each expressing a different TCR, with the antigen.

In another aspect, the present disclosure provides a composition comprising an antigen identified by a method provided herein. In some embodiments, the composition is a pharmaceutical composition comprising a pharmaceutically acceptable carrier.

In another aspect, the present disclosure provides a compartment comprising: a TCR-expressing cell comprising a T-cell receptor (TCR); and an antigen-presenting cell (APC) associated with a catch agent, wherein the catch agent is capable of binding to a polypeptide secreted by the TCR-expressing cell, and wherein the catch agent is (i) a soluble catch agent having specificity to both a cell surface protein of the APC and the polypeptide, or (ii) an exogenously expressed protein having specificity to the polypeptide. In some embodiments, the exogenously expressed protein is a membrane-bound protein. In some embodiments, the membrane-bound protein is a receptor. In some embodiments, the soluble catch agent is (i) a molecule expressed and/or secreted by the APC or (ii) a molecule supplied within the compartment. In some embodiments, the soluble catch agent has specificity to a cell surface protein of the APC and the polypeptide. In some embodiments, the soluble catch agent binds to both a cell surface protein of the APC and the polypeptide. In some embodiments, the cell surface protein is an endogenous protein or an exogenous protein of the APC. In some embodiments, the soluble catch agent is an antibody or a fragment thereof. In some embodiments, the antibody is a bispecific antibody (BsAb). In some embodiments, the compartment comprises a plurality of compartments, each compartment of the plurality of compartments comprising a TCR-expressing cell and an APC. In some embodiments, the compartment comprises a single APC.

In some embodiments, the compartment further comprises a plurality of polymerizable or gellable polymers and/or monomers. In some embodiments, the plurality of polymerizable or gellable polymers and/or monomers are polymerized or gelled.

In another aspect, the present disclosure provides a compartment comprising: a TCR-expressing cell comprising a T-cell receptor (TCR); and an antigen-presenting cell (APC) associated with a solid support, which solid support is further associated with a catch agent that is capable of binding to a polypeptide secreted by the TCR-expressing cell. In some embodiment, the solid support is modified with an antibody or fragment thereof, which antibody binds to a cell surface protein of the APC. In some embodiment, the catch agent is associated with the solid support via a crosslinker or an affinity binding pair. In some embodiment, the solid support is a bead or a hydrogel particle.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure”, “Fig.”, and “FIGURE” herein) of which:

FIG. 1 shows schematic representations of CD8+ or CD4+ T cell activation by an antigen-presenting cell (APC).

FIG. 2 shows an example of the methods described herein, where an activated T cell secretes cytokines captured by an APC comprising an antigen that can be recognized by the T cell.

FIG. 3A and FIG. 3B show an example scheme of antigen identification described herein.

FIG. 4 shows example microfluidic channels used for generating compartments (e.g., droplets) described herein.

FIG. 5 shows example results of cell viability in reactions (e.g., the APC and T cell interaction) performed in bulk or in emulsion with or without T cell activation.

FIG. 6 shows example flow cytometry results of detecting IL-2 captured by APCs after performing the reactions in bulk or in emulsion with or without T cell activation.

FIG. 7 shows an example of the methods described herein, where an activated T cell secretes cytokines captured by an APC comprising an antigen that can be recognized by the T cell.

FIG. 8 shows an example of the methods described herein, where the APC is directly linked to a catch agent.

FIGS. 9A-9F show an example of labeling APCs with cytokine capturing antibody for identification of T cell antigens. FIG. 9A shows a summary method for labeling APCs with a cytokine capturing antibody to detect antigen-specific T cells along with their cognate antigen.

FIG. 9B shows that biotinylated 293T and HeLa cells were stained positive for Strep-AF488 versus control cells. FIG. 9C shows that HeLa cells were highly specifically labeled with cytokine capturing antibody. The detection agent used in this experiment is clone M1 detection antibody. FIG. 9D shows that HeLa cells were labeled with cytokine capturing antibody. The detection agent used in this experiment is clone B27 detection antibody. The B27 detection clone appeared to be less sensitive to detection of IFNγ when bound to the 4S.B3 clone. FIG. 9E shows that 293T cells were labeled with cytokine capturing antibody. The detection agent used in this experiment is clone M1 detection antibody. There was an increased background labeling of 293T cells: non-biotinylated control cells showed some positive detection of IFNγ when cells were treated with Biotin-αIFNγ+IFNγ. FIG. 9F shows that 293T cells were labeled with cytokine capturing antibody. The detection agent used in this experiment is clone B27 detection antibody. The B27 detection clone appeared to be less sensitive to detection of IFNγ when bound to the 4S.B3 clone.

FIGS. 10A-10F show an example of a two-color labeling assay of WT vs. HLA-A02:01 presenting APCs to demonstrate specific labeling of APCs in co-culture with a model TCR T cell. FIG. 10A shows that DKO T cells electroporated with model TCR for NY-ESO peptide were stained with tetramer and CD3 to confirm expression of the model TCR at the surface.

FIG. 10B shows input ratio of K562-A2:K562-WT APCs in co-culture. K562-A2 cells were labeled with cell trace violet (shown in black) while K562-WT cells were labeled with cell trace far red (shown in gray). FIG. 10C shows percent IFNγ positive cells in various cell culture control conditions. IFNγ was specifically produced when APCs pulsed with model peptide were co-cultured with model TCR T cells. FIG. 10D shows percent IFNγ positive cells in cell culture conditions where the ratio of K562-A2 pulsed with NY-ESO:K562-WT is 1:100 or 1:1000. FIG. 10E shows that gating out T cells, the ratio of K562-A2:K562-WT cells was 1:1,000 within total cells, versus about 1:50 within all IFNγ+ cells. FIG. 10F shows fold enrichment of K562-A2 cells within the IFNγ positive cells when cultured at ratios of 1:100 versus 1:1,000 with K562-WT cells.

### DETAILED DESCRIPTION OF THE INVENTION

While various embodiments of the present disclosure have been shown and described herein, it will be obvious to those skilled in the art that such embodiments are provided by way of example only. Numerous variations, changes, and substitutions may occur to those skilled in the art without departing from the present disclosure. It should be understood that various alternatives to the embodiments of the present disclosure described herein may be employed.

In this disclosure, the use of the singular includes the plural unless specifically stated otherwise. Also, the use of “or” means “and/or” unless stated otherwise. Similarly, “comprise,” “comprises,” “comprising,” “include,” “includes,” and “including” are not intended to be limiting.

### Definitions

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polynucleotide,” “nucleic acid,” and “oligonucleotide” can be used interchangeably. They refer to a polymeric form of nucleotides. A polynucleotide can be of various lengths. They can be deoxyribonucleotides, ribonucleotides, or a combination thereof, or analogs thereof. A polynucleotide may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups. Polynucleotides may have any three-dimensional structure, and may perform various functions. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), circular RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotides may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs are compatible with natural and mutant polymerases for de novo and/or amplification synthesis.

The term “peptide,” as used herein, refers to a polymer of amino acids and which are joined together through amide bonds and is alternatively referred to as a “polypeptide”. In the context of this specification it should be appreciated that the amino acids may be the L-optical isomer or the D-optical isomer. Peptides are two or more amino acid monomers long, and often can be more than 20 amino acid monomers long. A polypeptide can be linearly unstructured or folded in three-dimensional structure. A structured polypeptide can be a protein. In some cases, a peptide is a neoantigen peptide. In some cases, a peptide is a tumor-associated antigen peptide.

The term “antigen,” as used herein, refers to an agent comprising an epitope against which an immune response can be be generated. An antigen can be a molecule which, optionally after processing, can induce an immune reaction. An antigen can be a protein, a peptide, a polysaccharide, a nucleic acid (e.g., RNA and DNA), or a nucleotide.

The term “neoantigen,” as used herein, refers to a class of tumor antigens that arises from a tumor-specific mutation(s) which alters the amino acid sequence of genome encoded proteins.

The term “sequence,” as used herein, can refer to a polypeptide sequence or a polynucleotide sequence. A polynucleotide sequence can be DNA or RNA; can be linear, circular or branched; and can be either single-stranded or double-stranded. A sequence can be mutated. A sequence can be of any length, for example, between 2 and 1,000,000 or more amino acids or nucleotides in length (or any integer value there between or there above), e.g., between about 100 and about 10,000 nucleotides or between about 200 and about 500 amino acids or nucleotides. The sequence of a nucleic acid can encompass the actual sequence and a reverse complement sequence of the sequence.

The term “compartment,” as used herein, refers to a compartment (e.g. a microfluidic channel, a well, a microwell, a hole, a tube, a capsule, or a droplet) in which a biochemical reaction (e.g., cell and cell interaction, target protein and antibody binding, nucleic acid hybridization, or primer extension) may occur. The terms “vessel” and “compartment” can be used interchangeably. The vessel or compartment may be solid-walled (when the boundary of the vessel or compartment is a solid such as glass, plastic, or polydimethylsiloxane (PDMS)) or liquid-walled (when the boundary of the vessel or compartment is a liquid such as oil). Solid-walled vessels may contain a solid scaffold, which is a continuous solid that connects all the vessels. The volume of the compartment may be as large as 1 mL or as small as 1 picoLiter. In some embodiments, the median size of the compartments of a plurality of compartments is from 1 to 10 picoLiter, from 10 to 100 picoLiter, from 100 picoLiter to 1 nanoLiter, from 1 to 10 nanoLiter, from 10 to 100 nanoLiter, from 100 nanoLiter to 1 microLiter, from 1 to 10 microLiter, from 10 to 100 microLiter, or from 100 to 1000 microLiter. The volume of the aqueous content in the compartment can be smaller than or about equal to the volume of the compartment. In some embodiments, the median volume of the aqueous content in the compartments is 1 microLiter or less.

The term “droplet,” as used herein, refers to a volume of liquid. An “emulsion” refers to a dispersion of minute droplets of a first liquid in a second liquid in which the first liquid is not soluble or miscible in the second liquid. Examples of emulsions include water-in-oil emulsion, water-in-oil-in-water emulsion, or water in a lipid layer (liposome) emulsion. As used herein: “water-in-oil emulsion” refers to a water-in-oil mixture in which the oil forms a continuous phase and the water is in discontinuous droplets. In some embodiments, droplets can be of uniform size or heterogeneous size. In some embodiments, the median diameter of the droplets in a plurality of droplets can range from about 0.001 μm to about 1 mm. In some embodiments, the median volume of the droplets in a plurality of droplets can range from 0.01 nanoLiter to 1 microLiter.

The term “partition,” as used herein, can be a verb or a noun. When used as a verb (e.g., “to partition,” or “partitioning”), the term generally refers to the fractionation (e.g., subdivision) of a species or sample between vessels that can be used to sequester one fraction (or subdivision) from another. Such vessels are referred to using the noun “partition.” Partitioning may be performed, for example, using microfluidics, dispensing, vortexing, and the like. A partition may be, for example, a well, a microwell, a hole, a droplet (e.g., a droplet in an emulsion), a continuous phase of an emulsion, a test tube, a spot, a capsule, a bead, a surface of a bead in dilute solution, or any other suitable container for sequestering one fraction of a sample from another. A partition may also comprise another partition. A water-in-oil emulsion can be created by using microfluidics or by physical agitation of a mixture of aqueous phase and an oil phase, optionally in the presence of a surfactant.

The terms “enriching,” “isolating,” “separating,” “sorting,” “purifying,” “selecting” or equivalents thereof can be used interchangeably herein and refer to obtaining a subsample with a given property from a sample. For example, they can refer to obtaining a cell population or cell sample that contains at least about 30%, 40%, 50%, 55%, 60%, 65%, 70%, 75%, 80%, 85%, 90%, 95%, or 100% of the desired cell lineage or a desired cell having a certain cell phenotype, e.g., expressing a certain cell marker or not expressing a certain cell marker gene characteristic of that cell phenotype.

The term “T cell epitope,” as used herein, refers to a peptide sequence, presented by MHC receptors on antigen-presenting cells and other nucleated cells. In some cases, antigen or candidate antigen can be used to refer to the T cell epitope. The T cell epitopes can be peptides derived from antigens and recognized by the T-cell receptor (TCR) when bound to MHC molecules. The T cell epitopes can stimulate immune system activity. The T cell epitopes may be short peptide sequences, e.g., ranging from 8-11 amino acids in length on MHC I complexes and 15-24 amino acids on MHC II complexes.

The term “antibody fragment,” as used herein, refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single domain antibodies or sdAb (e.g., VL, VH or VHH), single-chain antibody molecules (e.g., scFv), and multispecific antibodies formed from antibody fragments. In some embodiments, the antibodies are single-chain antibody fragments comprising a variable heavy chain region and/or a variable light chain region, such as scFv.

The term “exogenous,” as used herein, refers to a substance (e.g., a molecule) present in cells or organisms other than its own native source. On the other hand, the term “endogenous” refers to a substance that is native to the cells or organisms.

The term “exogenously expressing” or “exogenously expressed” refers to an expression of a polypeptide from an exogenous polynucleotide sequence (e.g., a polynucleotide sequence not derived or originated from the host cell) introduced to the host cell. An exogenous protein can be a protein expressed by an exogenous polynucleotide sequence that is not derived or originated from the host cell.

The term “associate,” as used herein, refers to making an association or connection. The association can be covalent or non-covalent. The association can be direct or indirect (e.g., via some intermediate such as a solid support). The association can be a stable interaction, for example, an interaction having a dissociation rate less than about 10⁻³, 10⁻⁴ or 10⁻⁵ per second. The term “associated with” can be used interchangeably with linked to, attached to, bound to, crosslinked to, or immobilized on. In some cases, the APC described herein is associated with a catch agent described herein.

The term “secreted” or “secreted protein,” as used herein, refers to a molecule or protein produced or expressed intracellularly and secreted from the cell to an extracellular environment.

The term “expression vector,” as used herein, refers to a vector comprising a recombinant polynucleotide comprising expression control sequences operatively linked to a nucleotide sequence to be expressed. An expression vector can comprise sufficient cis-acting elements for expression; other elements for expression can be supplied by the host cell or in an in vitro expression system. Expression vectors can include, for example, cosmids, plasmids (e.g., naked or contained in liposomes) and viruses (e.g., lentiviruses, retroviruses, adenoviruses, and adeno-associated viruses) that incorporate the recombinant polynucleotide.

The term “functional fragment,” as used herein, refers to a polypeptide which has at least one functional characteristic of an intact (e.g., native, wild-type or normal) protein as described herein, such as a binding activity, a signaling activity and/or ability to stimulate a cellular response.

### Overview

The present disclosure provides compositions and methods for identifying one or more antigens recognized by T-cell receptors (TCRs). The compositions and methods provided herein can be used to deorphanize a TCR. The compositions and methods provided herein can be used to screen (e.g., select or enrich) for antigen-presenting cells (APCs) expressing the one or more antigens recognized by a TCR based on a soluble factor (e.g., a cytokine) secreted by the cell expressing the TCR, which can be physically captured by the APCs. The ACPs expressing the one or more antigens can be sequenced to identify one or more nucleic acid sequences encoding the one or more antigens in order to identify the one or more antigens.

The compositions and methods provided herein can allow high-throughput, cost effective and sequencing based identification of antigen(s) recognized by a TCR.

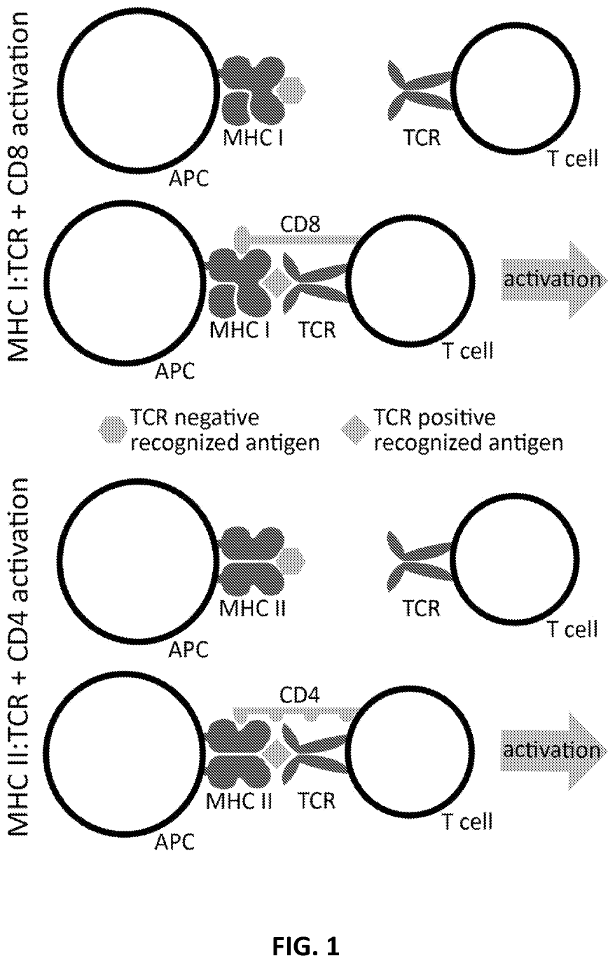

Engagement of TCRs with T cell epitopes (e.g., target peptides) presented on MHCs/HLAs on APCs can lead to the first signal in antigen-specific T cell activation (FIG. 1). Antigens can be processed by APCs and a corresponding peptide product can be displayed on the MHC, accessible to the TCRs on T cells. If the T cell epitope is not recognized by the TCR, no further interaction may occur. However, if a TCR recognizes the peptide-MHC complex, an antigen-specific activation pathway can be initiated, leading to the secretion of cytokines and the upregulation of surface markers by the T cell. The TCR-peptide-MHC interaction can occur in two different classes. MHC class I can interact with T cells that express the CD8 co-receptor (e.g., CD8+ T cells), while MHC class II can interact with T cells that express the CD4 co-receptor (e.g., CD4+ T cells). Both of these co-receptors can assist the T cells in communicating with the APCs.

Activation of a T cell through the TCR can lead to an internal signaling cascade that can induce cytokine secretion (e.g., IL-2, IFN-γ, TNFα, etc.). Accordingly, detection of cytokine secretion after stimulus can be evidence of T cell activation by that given stimulus. However, antigen information may not be obtained by detecting the secreted cytokines captured by the T cell.

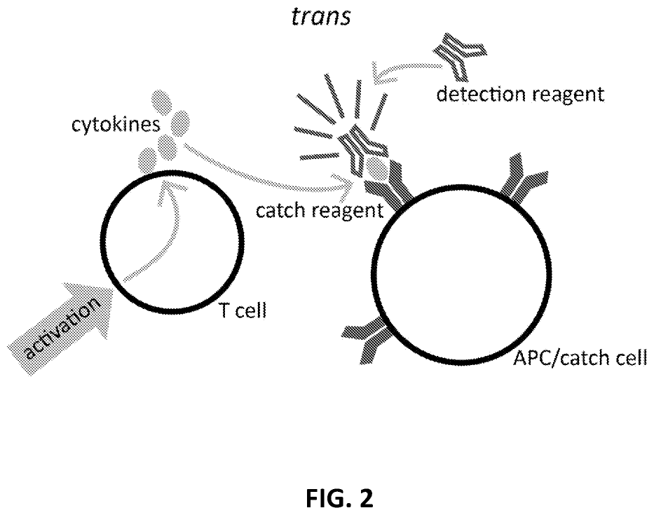

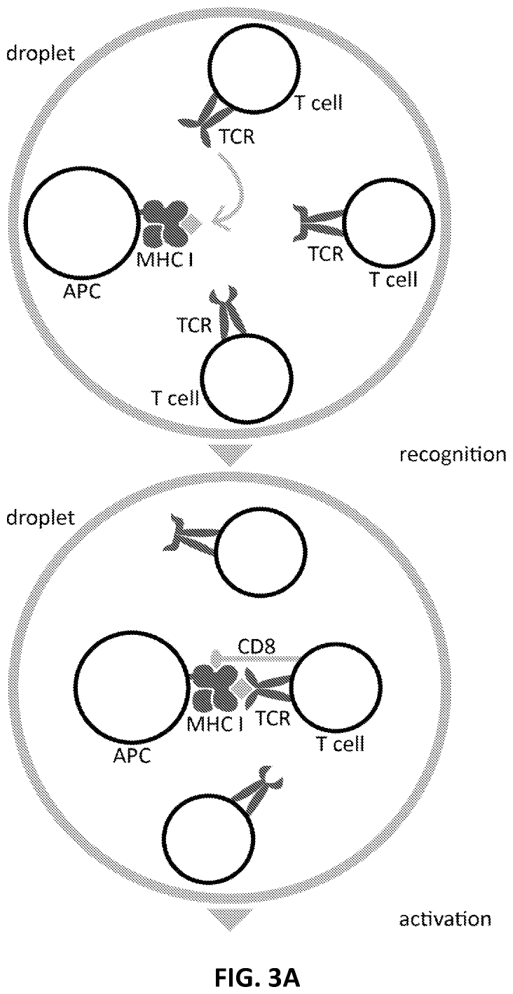

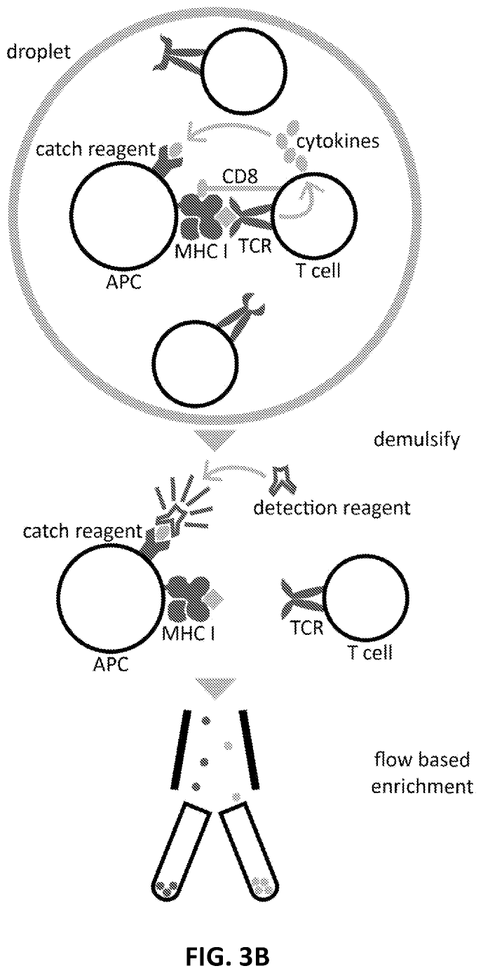

The present disclosure provides methods of labeling an APC recognized by a TCR with a polypeptide (e.g., a cytokine) secreted by a TCR-expressing cell, where a catch agent associated with the APC other than the TCR-expressing cell (e.g., a T cell) binds to the secreted polypeptide, thus catching the polypeptide “in trans” (e.g., trans cytokine catch). A second detection agent (or detection reagent) can be used to bind the captured polypeptide. The detection agent can provide an observable readout to indicate the presence of the caught polypeptide on the surface of the APC using assays such as flow cytometry. An example of the methods is shown in FIG. 2. In this figure, the APC, T cell and cytokines are used as an example, but the APC and the T cell can be other types of cells described herein and the cytokines can be other secreted polypeptides described herein. To deorphanize a TCR, multiple trans cytokine catch assays may be performed, where a plurality of APCs can be prepared, each expressing (in some cases, over-expressing) a different candidate antigen. If the APC catches the cytokine, it may indicate that the antigen expressed in the APC may be the antigen recognized by the T cell (e.g., the antigen contains the T cell epitope recognized by the T cell). The cytokine may not be secreted by a T cell recognizing an APC expressing another antigen. The methods can be performed by carrying out parallel trans cytokine catch assays in different compartments (e.g., wells or droplets), where there is only one or a few antigens expressed in the APCs in one compartment. An example method to perform multiple cytokine catch assays in a massively parallelized setting can be through the use of microfluidics such as droplet microfluidics, where a single droplet can act as an individual reaction chamber for a single assay (FIG. 3A and FIG. 3B). The TCR-expressing cells may not be activated or contacted with any APCs prior to being co-partitioned with the APC. The TCR-expressing cells may be non-activated cells. The APCs may not have been contacted with any TCR-expressing cells prior to being co-partitioned with the TCR-expressing cell(s). The method may not be limited to be performed in individual droplets. The APC, the T cell, and catch agent can be contained in the same reaction chamber such that the cytokine secreted by the T cell may be caught by the APC. The detection of cytokines can occur outside of the droplets (e.g., after the droplets are demulsified) after the catch process has been completed. General methods of making droplets, such as water-in-oil emulsions, include but are not limited to, microfluidics, vortexing, or shaking. Any of these methods can be used to generate the reaction chambers (also referred to as “compartments”) described herein. Performing the trans cytokine catch assay in droplets may allow for screening a single APC and antigen against an excess of T cells. In some cases, it is possible to screen against multiple T cells at the same time, since with the T cells being polyclonal to each other, only one of the TCRs can match with the WIC-peptide complex. The identified antigens can be used to prepare pharmaceutical compositions for treating a disease. The identified antigens can also be used to identify additional TCRs that can target the identified antigens.

FIG. 3A, top panel, shows an example compartment (e.g., a droplet) comprising a single APC and three different T cells expressing three different TCRs. The T cells can be non-activated T cells. Among the three T cells, one of the TCR can recognize the antigen presented by the APC. FIG. 3A, bottom panel, shows that the T cell having the TCR that recognizes the antigen can bind to the antigen complexed with the MHC molecule of the APC and the T cell can be activated. FIG. 3B, top panel, shows that the activated T cell can secrete cytokines, which can then be captured by the catch agent associated with the APC. FIG. 3B, bottom panel, shows that the cells can be released from the compartment (e.g., the emulsion can be demulsified) and the cytokines captured by the catch agent associated with the APC can be contacted with a detection agent. The APCs bound with cytokines can be detected or selected by flow-based enrichment (e.g., magnetic or flow cytometry). The magnetic or flow cytometry can comprise magnetic-activated cell sorting (MACS) or fluorescence-activated cell sorting (FACS). The detection agent may be co-partitioned in the compartment and thus may be contacted with the cytokine captured by the catch agent before the cells are released from the compartment.

### T-Cell Receptors (TCRs)

The present disclosure provides compositions and methods for identifying antigens or antigen-presenting cells (APCs) presenting the antigens that can be recognized by a T-cell receptor (TCR). The TCR can be expressed by a TCR-expressing cell such as a T cell.

The TCR can be used to confer the ability of T cells to recognize antigens (e.g., T cell epitopes) associated with various cancers or infectious organisms. The TCR can be made up of both an alpha (α) chain and a beta (β) chain or a gamma (γ) and a delta (δ) chain. The proteins which make up these chains can be encoded by DNA, which employs a unique mechanism for generating the tremendous diversity of the TCR. This multi-subunit immune recognition receptor can associate with the CD3 complex and bind peptides presented by the MHC class I and II proteins on the surface of antigen-presenting cells (APCs). Binding of a TCR to the antigenic peptide on the APC can be a central event in T-cell activation, which occurs at an immunological synapse at the point of contact between the T cell and the APC.

The TCR may recognize the T cell epitope in the context of an major histocompatibility complex (MHC) class I molecule. MHC class I proteins can be expressed in all nucleated cells of higher vertebrates. The MHC class I molecule is a heterodimer composed of a 46-kDa heavy chain which is non-covalently associated with the 12-kDa light chain β-2 microglobulin. The human MHC is also called the human leukocyte antigen (HLA) complex. In humans, there are several MHC alleles, such as, for example, HLA-A2, HLA-A1, HLA-A3, HLA-A24, HLA-A28, HLA-A31, HLA-A33, HLA-A34, HLA-B7, HLA-B45 and HLA-Cw8. In some embodiments, the MHC class I allele is an HLA-A2 allele, which in some populations is expressed by approximately 50% of the population. In some embodiments, the HLA-A2 allele can be an HLA-A*0201, *0202, *0203, *0206, or *0207 gene product. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203.

In some embodiments, the TCR may recognize the T cell epitope in the context of an MHC class II molecule. MHC class II proteins can be expressed in a subset of APCs. In humans, there are several MHC class II alleles, such as, for example, HLA-DR1, HLA-DR3, HLA-DR4, HLA-DR7, HLA-DR52, HLA-DQ1, HLA-DQ2, HLA-DQ4, HLA-DQ8 and HLA-DPI. In some embodiments, the MHC class II allele is an HLA-DRB1*0101, an HLA-DRB*0301, an HLA-DRB*0701, an HLA-DRB*0401 or an HLA-DQB1*0201 gene product.

The TCR chains can comprise a variable domain (or variable region) and a constant domain (or constant region). A full-length constant domain/region can comprise an extracellular portion (referred to as “extracellular constant domain” herein), a hinge region, a transmembrane region, and a cytoplasmic tail. In various embodiments, a constant domain can be a full-length constant domain or a portion thereof, for example, the extracellular constant domain. The variable domain of TCRα and δ chains is encoded by a number of variable (V) and joining (J) genes, while TCRβ and γ chains are additionally encoded by diversity (D) genes. During VDJ recombination, one random allele of each gene segment is recombined with the others to form a functional variable domain. Recombination of the variable domain with a constant gene segment can result in a functional TCR chain transcript. Additionally, random nucleotides may be added and/or deleted at the junction sites between the gene segments. This process can lead to strong combinatorial (depending on which gene regions will recombine) and junctional diversity (depending on which and how many nucleotides will be added/deleted), resulting in a large and highly variable TCR repertoire, which can ensure the identification of a plethora of antigens. Additional diversity can be achieved by the pairing (also referred to as “assembly”) of α and β or γ and δ chains to form a functional TCR. By recombination, random insertion, deletion and substitution, the small set of genes that encode the T cell receptor has the potential to create between 10¹⁵ and 10²⁰ TCR clonotypes. As used herein, a “clonotype” refers to a population of immune cells that carry an identical immunoreceptor. For example, a clonotype refers to a population of T cells that carry an identical TCR, or a population of B-cells that carry an identical BCR (or antibody). “Diversity” in the context of immunoreceptor diversity refers to the number of immunoreceptor (e.g., TCR, BCR and antibody) clonotypes in a population. The higher diversity in clonotype may indicate higher diversity in cognate pair combination.

Each TCR chain can contain three hypervariable loops in its structure, termed complementarity determining regions (CDR1-3). CDR1 and 2 are encoded by V genes and may be required for interaction of the TCR with the MHC complex. CDR3, however, is encoded by the junctional region between the V and J or D and J genes and therefore can be highly variable. CDR3 may be the region of the TCR in direct contact with the peptide antigen. CDR3 can be used as the region of interest to determine T cell clonotypes. The sum of all TCRs by the T cells of one individual is termed the TCR repertoire or TCR profile. The TCR repertoire can change with the onset and progression of diseases. Therefore, determining the immune repertoire status under different disease conditions, such as cancer, autoimmune, inflammatory and infectious diseases may be useful for disease diagnosis and prognosis.

The TCR may be a full-length TCR as well as antigen-binding portion or antigen-binding fragment (also called MHC-peptide binding fragment) thereof. In some embodiments, the TCR is an intact or full-length TCR. In some embodiments, the TCR is an antigen-binding portion that is less than a full-length TCR but that binds to a specific antigenic peptide bound to an MHC molecule, i.e., an MHC-peptide complex. An antigen-binding portion or fragment of a TCR can contain only a portion of the structural domains of a full-length or intact TCR, but yet is able to bind the epitope (e.g., MHC-peptide complex) to which the full TCR binds. In some cases, an antigen-binding portion or fragment of a TCR contains the variable domains of a TCR, such as variable a chain and variable R chain of a TCR, sufficient to form a binding site for binding to a specific MHC-peptide complex, such as generally where each chain contains three complementarity determining regions. Polypeptides or proteins having a binding domain which is an antigen-binding domain or is homologous to an antigen-binding domain are included.

### TCR-Expressing Cells

The TCR-expressing cell can be of various cell types. The TCR-expressing cell can be a cytokine-secreting cell. The TCR-expressing cell can express a TCR on its cell surface. The TCR-expressing cell can express an endogenous TCR or an exogenous TCR. The TCR-expressing cell can be a cell comprising a recombinant nucleic acid encoding a TCR. The TCR-expressing cell can secrete a polypeptide (e.g., a cytokine) upon interaction with antigen presented by an antigen-presenting cell (APC) described herein or upon activation by the APC. The TCR-expressing cell may not secrete the same cytokine or secrete very low amount of the same cytokine when the TCR-expressing cell has not been activated. For example, the TCR-expressing cell may not secrete the same cytokine or secrete very low amount of the same cytokine when the TCR-expressing cell has not been interacted with the antigen presented by the APC or has not been activated by the APC.

The polypeptide that is secreted by the TCR-expressing cell can be endogenous or exogenous of the TCR-expressing cell. For example, the TCR-expressing cell may be a T cell, and the polypeptide secreted can be a cytokine that is endogenous to the T cell. The TCR-expressing cell can be a CD4+ T cell, a CD8+ T cell, or a CD4+/CD8+ T cell. In some cases, the TCR-expressing cell may be other types of cells that may not endogenously express the cytokine, but the TCR-expressing cell may be engineered to express and secrete an exogenous protein (e.g., a cytokine that can be secreted or other proteins that can be secreted). The gene encoding such exogenous protein may be under the control of a transcription factor which is regulated by TCR signaling. Examples of these transcription factors include, but are not limited to, AP-1, NFAT, NF-kappa-B, Runx1, Runx3, etc. The exogenous protein can be a naturally secreted protein such as cytokines, chemokines, antibodies, extracellular enzymes, or a naturally non-secreted protein (such as a fluorescent protein or any naturally intracellular protein of human or non-human origin) engineered to be secreted. A naturally non-secreted protein can be engineered to be secreted by appending a signal peptide to the N-terminus of the protein to be secreted, this signal peptide sequence can be derived from naturally secreted proteins such as IgK, tissue plasminogen activator (tPA), secreted alkaline phosphatase (SEAP), or can be a synthetic sequence. The synthetic sequence may be a consensus sequence of multiple naturally secreted proteins. Some examples of the signal peptides include but are not limited to:

MWWRLWWLLLLLLLLWPMVWA,
METDTLLLWVLLLWVPGSTG,
MDMRVPAQLLGLLLLWLRGARC,
MPLLLLLPLLWAGALA,
MDAMKRGLCCVLLLCGAVFVSPS,
MLLLLLLLLLLALALA,
and
MLLLLLLLGLRLQLSLG.

The TCR-expressing cell can be a T cell. The TCR-expressing cell such as T cells can be obtained from a subject (e.g., primary T cells). The subject can be a healthy subject or a subject having a disease or a condition. The subject can be a cancer patient. The TCR-expressing cell may be obtained from any sample described herein. For example, the sample can be a peripheral blood sample. The peripheral blood cells can be enriched for a particular cell type (e.g., mononuclear cells; red blood cells; CD4+ cells; CD8+ cells; immune cells; T cells, NK cells, or the like). The peripheral blood cells can also be selectively depleted of a particular cell type (e.g., mononuclear cells, red blood cells, CD4+ cells, CD8+ cells, immune cells, T cells, NK cells, or the like). The TCR-expressing cell can be an autologous T cell or an allogeneic T cell.

In some cases, the TCR-expressing cell can be obtained from a tissue sample comprising a solid tissue, with non-limiting examples including a tissue from brain, liver, lung, kidney, prostate, ovary, spleen, lymph node (including tonsil), thyroid, thymus, pancreas, heart, skeletal muscle, intestine, larynx, esophagus, and stomach. Additional non-limiting sources include bone marrow, cord blood, tissue from a site of infection, ascites, pleural effusion, spleen tissue, and tumors. In some embodiments, T cell lines may be used. In some embodiments, the cell can be derived or obtained from a healthy donor, from a patient diagnosed with cancer or from a patient diagnosed with an infection. In some embodiments, the cell is part of a mixed population of cells which present different phenotypic characteristics.

The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects, T cell lines may be used. T cells can be helper T cells, cytotoxic T cells, memory T cells, regulatory T cells, natural killer T cells, alpha beta T cells, or gamma delta T cells. In certain aspects of the present disclosure, T cells can be obtained from a unit of blood collected from a subject using a variety of techniques, such as Ficoll™ separation. Cells from the circulating blood of an individual can be obtained by apheresis. The apheresis product may contain lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. In some cases, the cells can be washed with phosphate buffered saline (PBS). The wash solution may lack calcium or magnesium or other divalent cations. Initial activation steps in the absence of calcium can lead to magnified activation. A washing step may be accomplished by methods such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In an aspect, T cells are isolated from peripheral blood lymphocytes or tissues by lysing the red blood cells and depleting the monocytes, for example, by centrifugation through a PERCOLL™ gradient or by counterflow centrifugal elutriation. When isolating T cells from tissues (e.g., isolating tumor-infiltrating T cells from tumor tissues), the tissues made be minced or fragmented to dissociate cells before lysing the red blood cells or depleting the monocytes. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least or equal to about 1, 2, 3, 4, 5, or 6 hours. In yet another aspect, the time period is 10 to 24 hours. In an aspect, the incubation time period is about 24 hours. Longer incubation times may be used to isolate T cells in any situation where there are few T cells as compared to other cell types, such as in isolating tumor infiltrating lymphocytes (TILs) from tumor tissue or from immunocompromised individuals. Further, use of longer incubation times can increase the efficiency of capture of CD8+ T cells. Thus, by simply shortening or lengthening the time T cells are allowed to bind to the anti-CD3/anti-CD28 beads and/or by increasing or decreasing the ratio of beads to T cells, subpopulations of T cells can be selected for or against at culture initiation or at other time points during the process. Additionally, by increasing or decreasing the ratio of anti-CD3 and/or anti-CD28 antibodies on the beads or other surface, subpopulations of T cells can be selected for or against at culture initiation or at other desired time points. In some cases, multiple rounds of selection can be used. In certain aspects, the selection procedure can be performed and the “unselected” cells (cells that may not bind to the anti-CD3/anti-CD28 beads) can be used in the activation and expansion process. “Unselected” cells can also be subjected to further rounds of selection.

Enrichment of a T cell population by negative selection can be accomplished with a combination of antibodies directed to surface markers unique to the negatively selected cells. An example method can be cell sorting and/or selection via negative magnetic immune adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be useful to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ, TNF-alpha, IL-17A, IL-2, IL-3, IL-4, GM-CSF, IL-10, IL-13, granzyme B, and perforin, or other molecules, e.g., other cytokines and transcription factors such as T-bet, Eomes, Tcf1 (TCF7 in human). Methods for screening for cell expression can be determined, e.g., by the methods described in PCT Publication No.: WO 2013/126712.

For isolation of a population of cells by positive or negative selection, the concentration of cells and surface (e.g., particles such as beads) can be varied. In certain aspects, the volume in which beads and cells are mixed together may be decreased (e.g., increase the concentration of cells) to ensure maximum contact of cells and beads. For example, in an aspect, a concentration of 2 billion cells/mL is used. In another aspect, a concentration of 1 billion cells/mL is used. In a further aspect, greater than 100 million cells/mL is used. In a further aspect, a concentration of cells of at least about 10, 15, 20, 25, 30, 35, 40, 45, or 50 million cells/mL is used. In some aspects, a concentration of cells of at least about 75, 80, 85, 90, 95, or 100 million cells/mL is used. In some aspects, a concentration of cells of at least about 125 or 150 million cells/mL can be used. Using high concentrations can result in increased cell yield, cell activation, and cell expansion. Further, use of high cell concentrations can allow more efficient capture of cells that may weakly express cell surface markers of interest, such as CD28-negative T cells, or from samples where there are many tumor cells present (e.g., leukemic blood, tumor tissue, etc.). Such populations of cells may have therapeutic value. For example, using high concentration of cells can allow more efficient selection of CD8+ T cells that may have weaker CD28 expression.

In some cases, lower concentrations of cells may be used. By significantly diluting the mixture of T cells and surface, interactions between the particles and cells can be minimized. This can select for cells that express high amounts of desired antigens to be bound to the particles. For example, CD4+ T cells can express higher levels of CD28 and can be more efficiently captured than CD8+ T cells in dilute concentrations. In some aspects, the concentration of cells used is at least about 5×10⁵/mL, 5×10⁶/mL, or more. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells can also be frozen after a washing step. The freeze and subsequent thaw step may provide a more uniform product by removing granulocytes and to some extent monocytes in the cell population. After the washing step that removes plasma and platelets, the cells may be suspended in a freezing solution. While many freezing solutions and parameters may be useful in this context, one method that can be used involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing Hespan and PlasmaLyte A. The cells can then be frozen to −80° C. and stored in the vapor phase of a liquid nitrogen storage tank. Cell may be frozen by uncontrolled freezing immediately at −20° C. or in liquid nitrogen. In certain aspects, cryopreserved cells are thawed and washed and allowed to rest for one hour at room temperature prior to use.

Also contemplated in the context of the present disclosure is the collection of blood samples or apheresis product from a subject at a time period prior to when cells (e.g., TCR-expressing cells) might be needed. In some cases, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Besides primary T cells obtained from a subject, the TCR-expressing cells may be cell-line cells, such as cell-line T cells. Examples of cell-line T cells include, but are not limited to, Jurkat, CCRF-CEM, HPB-ALL, K-T1, TALL-1, MOLT 16/17, and HUT 78/H9.

The TCR-expressing cell can be a T cell obtained from an in vitro culture. T cells can be activated or expanded in vitro by contacting with a tissue or a cell. See “Activation and Expansion” section. For example, the T cells isolated from a patient's peripheral blood can be co-cultured with cells presenting tumor antigens such as tumor cells, tumor tissue, tumorsphere, tumor lysate-pulsed APC or tumor mRNA-loaded APC. The cells presenting tumor antigens may be APC pulsed with or engineered to express a defined antigen, a set of defined antigens or a set of undefined antigens (such as tumor lysate or total tumor mRNA). For example, in the cases of presenting defined antigens, an APC can express one or more minigenes encoding one or more short epitopes (e.g., from 7 to 13 amino acids in length) with known sequences. An APC can also express two or more minigenes from a vector containing sequences encoding the two or more epitopes. In the cases of presenting undefined antigens, an APC can be pulsed with tumor lysate or total tumor mRNA. The cells presenting tumor antigens may be irradiated before the co-culture. The co-culture may be in media comprising reagents (e.g., anti-CD28 antibody) that may provide co-stimulation signal or cytokines. Such co-culture may stimulate (e.g., activate) and/or expand tumor antigen-reactive T cells. These cells may be selected or enriched using cell surface markers described herein (e.g., CD25, CD69, CD137). Using this method, tumor antigen-reactive T cells can be pre-enriched from the peripheral blood of the patient. These pre-enriched T cells can be used as the TCR-expressing cells in the methods described herein. The pre-enriched T cells (e.g., CD137+) may contain T cells that acquired marker (e.g., CD137) expression during the co-culture, and may also contain T cells that already express the marker at blood draw. The latter population may nevertheless be tumor reactive. This method can offer an easier alternative to isolating tumor-infiltrating lymphocytes (TILs) described.

The TCR-expressing cell can be a tumor-infiltrating lymphocyte (TIL), e.g., tumor-infiltrating T cells. A TIL can be isolated from an organ afflicted with a cancer. One or more cells can be isolated from an organ with a cancer that can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. One or more TILs can be from a brain, heart, liver, skin, intestine, lung, kidney, eye, small bowel, or pancreas. TILs can be from a pancreas, kidney, eye, liver, small bowel, lung, or heart. The one or more cells can be pancreatic islet cells, for example, pancreatic β cells. In some cases, a TIL can be from a gastrointestinal cancer. A TIL culture can be prepared a number of ways. For example, a tumor can be trimmed from non-cancerous tissue or necrotic areas. A tumor can then be fragmented to about 2-3 mm in length. In some cases, a tumor can be fragmented from about 0.5 mm to about 5 mm in size, from about 1 mm to about 2 mm, from about 2 mm to about 3 mm, from about 3 mm to about 4 mm, or from about 4 mm to about 5 mm. Tumor fragments can then be cultured in vitro utilizing media and a cellular stimulating agent such as a cytokine. In some cases, IL-2 can be utilized to expand TILs from a tumor fragment. A concentration of IL-2 can be about 6000 IU/mL. A concentration of IL-2 can also be about 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL. Once TILs are expanded, they can be subject to in vitro assays to determine tumor reactivity. For example, TILs can be evaluated by FACs for CD3, CD4, CD8, and CD58 expression. TILs can also be subjected to cocultured, cytotoxicity, ELISA, or ELISPOT assays. In some cases, TIL cultures can be cryopreserved or undergo a rapid expansion. A cell, such as a TIL, can be isolated from a donor of a stage of development including, but not limited to, fetal, neonatal, young and adult.

The TCR-expressing cells can be T cells, B cells, NK cells, macrophages, neutrophils, granulocytes, eosinophils, red blood cells, platelets, stem cells, iPSCs, or mesenchymal stem cells. In addition, the TCR-expressing cell can be a cell line cell. The cell line can be tumorigenic or artificially immortalized cell line. Examples of cell lines include, but are not limited to, CHO-K1 cells, HEK293 cells, Caco2 cells, U2-OS cells, NIH 3T3 cells, NSO cells, SP2 cells, CHO-S cells, DG44 cells, K-562 cells, U-937 cells, MRCS cells, IMR90 cells, Jurkat cells, HepG2 cells, HeLa cells, HT-1080 cells, HCT-116 cells, Hu-h7 cells, Huvec cells, and Molt 4 cells. The TCR-expressing cell can be an autologous T cell or an allogeneic T cell. The TCR-expressing cell can be a genetically modified or engineered cell. The TCR-expressing cell (e.g., the cell line cell) may be engineered to express a TCR.

### Activation and Expansion

The TCR-expressing cell can be a T cell. The T cell can be expanded or stimulated by contact with an agent that stimulates a CD3 TCR complex and a co-stimulatory molecule on the surface of the T cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T-cell. As non-limiting examples, T cell populations may be stimulated in vitro such as by contact with an anti-CD3 antibody or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. For example, the agents providing each signal may be in solution or coupled to a surface. The ratio of particles to cells may depend on particle size relative to the target cell. In further embodiments, the cells, such as T cells, are combined with agent-coated beads, the beads and the cells are subsequently separated, and then the cells are cultured. In an alternative embodiment, prior to culture, the agent-coated beads and cells are not separated but are cultured together. Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFβ, and TNF-α or any other additives for the growth of cells. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. The target cells can be maintained under conditions necessary to support growth, for example, an appropriate temperature (e.g., 37° C.) and atmosphere (e.g., air plus 5% CO₂). T cells that have been exposed to varied stimulation times may exhibit different characteristics. The T cell can be activated or expanded by co-culturing with tissue or cells. The cells used to activate T cells can be APC or artificial APC (aAPC).

In some cases, stimulation of T cells can be performed with antigen and irradiated, histocompatible APCs, such as feeder PBMCs. In some cases, cells can be grown using non-specific mitogens such as PHA and allogenic feeder cells. Feeder PBMCs can be irradiated at 40Gy. Feeder PBMCs can be irradiated from about 10 Gy to about 15 Gy, from about 15 Gy to about 20 Gy, from about 20Gy to about 25 Gy, from about 25 Gy to about 30 Gy, from about 30 Gy to about 35 Gy, from about 35 Gy to about 40 Gy, from about 40 Gy to about 45 Gy, from about 45 Gy to about 50 Gy. In some cases, a control flask of irradiated feeder cells only can be stimulated with anti-CD3 and IL-2.

### Antigen-Presenting Cells (APCs)

The present disclosure provides methods of identifying antigens presented by a plurality of antigen-presenting cells (APCs). Each APC of the plurality of APCs can comprise a different antigen. The antigen can be in complex with a MHC molecule. The antigen can be a peptide. The antigen can be a T cell epitope. In some cases, the plurality of APCs, each APC comprising a different antigen, can comprise at least about 5, 10, 20, 50, 100, 150, 200, 250, 300, 350, 400, 450, 500, 550, 600, 650, 700, 750, 800, 850, 900, 950, 1,000 or more APCs. The plurality of APCs can comprise at least about 5, 10, 20, 50, 100, 150, 200, 250, 300, 350, 400, 450, 500, 550, 600, 650, 700, 750, 800, 850, 900, 950, 1,000 or more different antigens. In some cases, the plurality of APCs, each APC comprising a different antigen, can comprise at least about 1,000, 2,000, 3,000, 4,000, 5,000, 6,000, 7,000, 8,000, 9,000, 10,000, 100,000, 1,000,000 or more APCs. The plurality of APCs can comprise at least about 1,000, 2,000, 3,000, 4,000, 5,000, 6,000, 7,000, 8,000, 9,000, 10,000, 100,000, 1,000,000 or more different antigens. The APCs comprising an antigen that can be recognized by the TCR-expressing cell described herein can activate (e.g., stimulate) the TCR-expressing cell. The activated TCR-expressing cell can secrete one or more polypeptides. The polypeptides can be endogenous or exogenous to the TCR-expressing cell. The polypeptide can be engineered to be secreted by the TCR-expressing cell. The activated TCR-expressing cell can secrete one or more cytokines.

The antigen can include a tumor-specific antigen, a tumor-associated antigen, an embryonic antigen on tumor, a tumor-specific membrane antigen, a tumor-associated membrane antigen, a growth factor receptor, a growth factor ligand, or any other type of antigen that is associated with a cancer. The tumor antigen can be a tumor-specific antigen (TSA). The term “TSA,” as used herein, refers to an antigen that is unique to tumor cells and does not occur on other cells in the body. The tumor antigen can be a tumor-associated antigen (TAA). The term “TAA,” as used herein, refers to an antigen that is not unique to a tumor cell and is also expressed on a normal cell. The expression of the antigen on the tumor can occur under conditions that enable the immune system to respond to the antigen. The TAA may be expressed at much higher levels on tumor cells. The TAA can be determined by sequencing a patient's tumor cells and identifying mutated proteins only found in the tumor. These antigens are referred to as “neoantigens.” The tumor antigen can be an epithelial cancer antigen, a prostate specific cancer antigen (PSA) or prostate specific membrane antigen (PSMA), a bladder cancer antigen, a lung cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, a colorectal cancer antigen, a lymphoma antigen, a B-cell lymphoma cancer antigen, a leukemia antigen, a myeloma antigen, an acute lymphoblastic leukemia antigen, a chronic myeloid leukemia antigen, or an acute myelogenous leukemia antigen. Examples of antigens include, but are not limited to, 1GH-IGK, 43-9F, 5T4, 791Tgp72, 9D7, acyclophilin C-associated protein, alpha-fetoprotein (AFP), α-actinin-4, A3, antigen specific for A33 antibody, ART-4, B7, Ba 733, BAGE, BCR-ABL, beta-catenin, beta-HCG, BrE3-antigen, BCA225, BING-4, BRCA1/2, BTAA, CA125, CA 15-3\CA 27.29\BCAA, CA195, CA242, CA-50, calcium activated chloride channel 2, CAGE, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CML6/6, CO-029, CTLA4, CXCR4, CXCR7, CXCL12, cyclin B, HIF-1a, colon-specific antigen-p (CSAp), CEA (CEACAMS), CEACAM6, c-Met, DAM, E2A-PRL, EGFR, EGFRvIII, EGP-1 (TROP-2), EGP-2, ELF2-M, Ep-CAM, EphA3, fibroblast growth factor (FGF), FGF-5, fibronectin, Flt-1, Flt-3, folate receptor, G250 antigen, Ga733VEpCAM, GAGE, gp100, GRO-β, H4-RET, HLA-DR, HM1.24, human chorionic gonadotropin (HCG) and its subunits, HMGB-1, hypoxia inducible factor (HIF-1), HSP70-2M, HST-2, HTgp-175, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, immature laminin receptor, insulin-like growth factor-1 (IGF-1), KC4-antigen, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MC1R, mCRP, MCP-1, mesothelin, MIP-1A, MIP-1B, MIF, MG7-Ag, MOV18, MUC1, MUC2, MUC3, MUC4, MUCSac, MUC13, MUC16, MUM-1/2, MUM-3, MYL-RAR, NB/70K, Nm23H1, NuMA, NCA66, NCA95, NCA90, NY-ESO-1, P polypeptide, p15, p16, p53, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pme117 prostatic acid phosphatase, PSA, PRAME, PSMA, P1GF, ILGF, ILGF-1R, IL-6, IL-25, RCAS1, RS5, RAGE, RANTES, Ras, T101, SAGE, SAP-1, 5100, SSX-2, survivin, survivin-2B, SDDCAG16, TA-90\Mac2 binding protein, TAAL6, TAC, TAG-72, Ig TCR, TLP, telomerase, tenascin, TRAIL receptors, TRP-1, TRP-2, TSP-180, TNF-α, Tn antigen, Thomson-Friedenreich antigens, tumor necrosis antigens, tyrosinase, VEGFR, ED-B fibronectin, WT-1, XAGE, 17-1A-antigen, complement factors C3, C3a, C3b, C5a, C5, an angiogenesis marker, bc1-2, bc1-6, and K-ras, an oncogene marker and an oncogene product. The antigen can also comprise infectious disease associated antigen or an autoimmune disease associated antigen.

The APC described herein can be used to present antigen to the TCR-expressing cell for antigen identification. The APC described herein may also be used to activate or expand cells such as T cells.

The APC can express a MEW molecule. The APC may be engineered to express an exogenous MHC molecule. The MEW molecule can be a MEW class I molecule or a MHC class II molecule. For example, the MHC molecule can be a HLA-A2, HLA-A1, HLA-A3, HLA-A24, HLA-A28, HLA-A31, HLA-A33, HLA-A34, HLA-B7, HLA-B45 or HLA-Cw8 molecule. For another example, the MHC molecule can be a HLA-DR1, HLA-DR3, HLA-DR4, HLA-DR7, HLA-DR52, HLA-DQ1, HLA-DQ2, HLA-DQ4, HLA-DQ8 or HLA-DPI molecule. The APC can present an antigen. The APC can comprise a catch agent described herein. For example, the catch agent can be a soluble protein associated with the cell surface of the APC. For another example, the catch agent can be a membrane-bound protein endogenously or exogenously expressed by the APC. The soluble catch agent can have specificity to both a cell surface protein of an APC and a polypeptide secreted by the activated TCR-expressing cell described herein. The soluble catch agent can bind to both the cell surface protein of the APC and the polypeptide. The cell surface protein can be various types of surface proteins. The cell surface protein may not be a marker exclusively expressed by the APC. For example, it may be a cell surface protein also expressed by the TCR-expressing cell, but as long as the APC expresses the cell surface protein, it can be used as a target for the soluble catch agent. The soluble catch agent can comprise an antigen binding domain that targets the cell surface protein. The antigen binding domain can be an antibody or a fragment thereof that targets the cell surface protein. Such antibody or a fragment thereof may be obtained from various commercial sources. Non-limiting examples of the cell surface protein include CD11b, CD11c, CD14, CD80, CD86, B7-1, B7-2, CD18, CD29, CD31, CD40, CD43, CD44, CD45, CD54, CD58, CD83, CD86, IFN-γ receptor, IL-2 receptor, ICAM-1, Fcγ receptor, CMRF-44, CMRF-56, DCIR, and DECTIN-1.

A nucleic acid molecule or sequence encoding an antigen can be delivered into the APC described herein. For example, the nucleic acid molecule or sequence encoding antigen can be transfected or transduced into the APC. The nucleic acid molecule can be deoxyribonucleic acid (DNA), ribonucleic acid (RNA), or a combination thereof. The nucleic acid molecule may comprise a modified nucleotide. The nucleic acid molecule may comprise an analog of a nucleic acid.

The nucleic acid molecule or sequence encoding an antigen can comprise a full coding sequence of a gene. The gene can be any gene in the genome of an organism. The nucleic acid molecule or sequence encoding an antigen can comprise a partial sequence or a fragment of a gene. The nucleic acid molecule or sequence encoding an antigen can be at least about 10, 20, 30, 40, 50, 60, 70, 80, 90, 100, 120, 150, 200, 220, 250, 300 or more nucleotides in length. The nucleic acid molecule encoding an antigen can be at least about 200, 300, 400, 500, 600, 700, 800, 900, 1,000, 2,000, 3,000, 4,000, 5,000, 6,000, 7,000, 8,000, 9,000, 10,000 or more nucleotides in length.

The nucleic acid molecule or sequence encoding an antigen can be delivered into the APC by a vector. The vector can comprise the nucleic acid sequence encoding the antigen. The vector may comprise a marker gene (e.g., GFP) for the purpose of determining vector titers and transfection or transduction efficiency. In some cases, the cells can be transfected or transduced at a low multiplicity of infection (MOI) such as at MOI of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 or more. In some cases, the cells can be transfected or transduced at MOI of at most about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5 or less. In some cases, a library of nucleic acid molecules encoding a plurality of different antigens can be delivered into a plurality of APCs such that an individual APC can express only one, two, three, four, five, six, seven, eight or more antigens. In some cases, a library nucleic acid molecules encoding a plurality of different antigens can be delivered into a plurality of APCs such that an individual APC can express at most about eight, seven, six, five, four, three, two or one antigen(s). The library of nucleic acid molecules encoding a plurality of different antigens can comprise a tiling array of nucleic acid fragments (e.g., fragments with overlapping sequences) of a gene.

The vector for delivering a nucleic acid (e.g., the nucleic acid encoding an antigen) can be various types of vectors. The vector described herein can be used to deliver nucleic acids encoding other exogenous proteins into a host cell. The vector can be a viral or non-viral vector. The vector can be a plasmid, a transposon (e.g., Sleeping Beauty, Piggy Bac), or a viral vector (e.g., adenoviral vector, AAV vector, retroviral vector and lentiviral vector). Additional examples of a vector include a shuttle vector, a phagemide, a cosmid and an expression vector. Non-limiting examples of plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some cases, a vector is a nucleic acid molecule as introduced into a host cell, thereby producing an engineered cell. A vector may include nucleic acid sequences that permit it to replicate in a recipient cell, such as an origin of replication. A vector may also include one or more selectable marker genes and other genetic elements. A vector can be a viral or a non-viral vector, such as a retroviral vector (including lentiviral vectors), an adenoviral vector including replication competent, replication deficient and gutless forms thereof, an adeno-associated virus (AAV) vector, a simian virus 40 (SV-40) vector, a bovine papilloma vector, an Epstein-Barr vector, a herpes vector, a vaccinia vector, a Moloney murine leukemia vector, a Harvey murine sarcoma virus vector, a murine mammary tumor virus vector, a Rous sarcoma virus vectors, a Baculovirus vector, and a nonviral plasmid.

In some embodiments, the vector is a self-amplifying RNA replicon, also referred to as self-replicating (m)RNA, self-replication (m)RNA, self-amplifying (m)RNA, or RNA replicon. The self-amplifying RNA replicon can be an RNA that can replicate itself. In some embodiments, the self-amplifying RNA replicon can replicate itself inside of a cell. In some embodiments, the self-amplifying RNA replicon encodes an RNA polymerase and a molecule of interest. The RNA polymerase may be a RNA-dependent RNA polymerase (RDRP or RdRp). The self-amplifying RNA replicon may also encode a protease or an RNA capping enzyme. In some embodiments, the self-amplifying RNA replicon vector is of or derived from the Togaviridae family of viruses known as alphaviruses which can include Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, South African Arbovirus No. 86, Semliki Forest virus, Middelburg virus, Chikungunya virus, Onyong-nyong virus, Ross River virus, Barmah Forest Virus, Getah Virus, Sagiyama virus, Bebaru virus, Mayaro virus, Una virus, Aura virus, Whataroa virus, Babanki virus, Kyzylagach virus, Highlands J Virus, Fort Morgan virus, Ndumu virus, Buggy Creek virus, and any other virus classified by the International Committee on Taxonomy of Viruses (ICTV) as an alphavirus. In some embodiments, the self-amplifying RNA replicon is or contains parts from an attenuated form of the alphavirus, such as the VEE TC-83 vaccine strain. In some embodiments, the self-amplifying RNA replicon vector is an attenuated form of the virus that allows for expression of the molecules of interests without cytopathic or apoptotic effects to the cell. In some embodiments, the self-amplifying RNA replicon vector has been engineered or selected in vitro, in vivo, ex vivo, or in silica for a specific function in the host cell, target cell, or organism. For example, a population of host cells harboring different variants of the self-amplifying RNA replicon can be selected based on the expression level of one or more molecules of interest (encoded in the self-amplifying RNA replicon or in the host genome) at different time point. In some embodiments, the self-amplifying RNA replicon can contain one or more sub-genomic sequence(s) to produce one or more sub-genomic polynucleotide(s). In some embodiments, the sub-genomic polynucleotides act as functional mRNA molecules for translation by the cellular translation machinery. A sub-genomic polynucleotide can be produced via the function of a defined sequence element (e.g., a sub-genomic promoter or SGP) on the self-amplifying RNA replicon that directs a polymerase to produce the sub-genomic polynucleotide from a sub-genomic sequence. In some embodiments, the SGP is recognized by an RNA-dependent RNA polymerase (RDRP or RdRp). In some embodiments, multiple SGP sequences are present on a single self-amplifying RNA replicon and can be located upstream of sub-genomic sequence encoding the antigen. In some embodiments, the nucleotide length or composition of the SGP sequence can be modified to alter the expression characteristics of the sub-genomic polynucleotide. In some embodiments, non-identical SGP sequences are located on the self-amplifying RNA replicon such that the ratios of the corresponding sub-genomic polynucleotides are different from instances where the SGP sequences are identical. In some embodiments, the location of the sub-genomic sequences and SGP sequences relative to one another and the genomic sequence itself can be used to alter the ratio of sub-genomic polynucleotides relative to one another.

Various methods of delivering (or introducing) and expressing genes into a cell can be used. In the context of an expression vector, the vector can be readily introduced into a host cell, e.g., APC. For example, the expression vector can be transferred into a host cell by physical, chemical, or biological methods. Physical methods for introducing a nucleic acid molecule into a host cell include, but are not limited to, calcium phosphate precipitation, lipofection, particle bombardment, microinjection, electroporation, and the like. Biological methods for introducing a nucleic acid molecule of interest into a host cell include, but are not limited to, the use of DNA and RNA vectors. Viral vectors such as retroviral vectors, lentiviral vectors, adenoviral vectors and adeno-associated viral vectors, can be used for delivering genes into mammalian cells, e.g., human cells. Chemical methods for introducing a nucleic acid molecule into a host cell can include colloidal dispersion systems, such as macromolecule complexes, nanocapsules, microspheres, beads, and lipid-based systems including oil-in-water emulsions, micelles, mixed micelles, and liposomes. An example colloidal system for use as a delivery vehicle in vitro and in vivo is a liposome (e.g., an artificial membrane vesicle). Other methods can include, but are not limited to, delivery of nucleic acids with targeted nanoparticles or other suitable sub-micron sized delivery system.

In the case where a non-viral delivery system is utilized, an example delivery vehicle is a liposome. The use of lipid formulations can be contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They can form spontaneously when phospholipids are suspended in an excess of aqueous solution. The lipid components can undergo self-rearrangement before the formation of closed structures and entrap water and dissolved solutes between the lipid bilayers. However, compositions that have different structures in solution than the normal vesicular structure are also encompassed. For example, the lipids may assume a micellar structure or merely exist as nonuniform aggregates of lipid molecules. Also contemplated herein can include lipofectamine-nucleic acid complexes.

The APC can be various types of cells. The APC can be an engineered cell. The APC may be an artificial APC (aAPC). The APC can be professional APC such as dendritic cell, macrophage, or B cell. The APC can be a monocyte or monocyte-derived dendritic cell. The APC can be non-professional APC such as a fibroblast, a thymic epithelial cell, a thyroid epithelial cell, a glial cell, a pancreatic beta cell, or a vascular endothelial cell.

An aAPC can express ligands for TCR and costimulatory molecules and can activate and expand T cells. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination thereof. A signal 1 can be an antigen recognition signal. For example, signal 1 can be ligation of a TCR by a peptide-WIC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof.

In some cases, an aAPC may be used to contact with a cell population for antigen identification. In some cases, an aAPC may be used to stimulate, activate and/or expand a cell population. In some cases, an aAPC may not induce allospecificity. An aAPC may not express HLA in some cases. An aAPC may be genetically modified to stably express an antigen. An aAPC may be genetically modified to stably express genes that can be used to activation and/or stimulation. For example, a K562 cell may be genetically modified to express an antigen. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can express a membranous form of anti-CD3 mAb, clone OKT3, membranous form of anti-CD28 mAb in addition to CD80 and CD83. In some cases, an engineered K562 cell can a catch agent described herein. For example, the K562 cell can be engineered to express a cytokine receptor such as an IL-2 receptor. The K562 can be associated with a soluble catch agent having specificity to both a cell surface protein of the K562 cell and the cytokine. For example, the K562 cell can be associated with a bispecific antibody having specificity to CD45 and IL-2.

An aAPC can be a bead. A spherical polystyrene bead can be coated with peptide-WIC complex and optionally antibodies against CD28 and be used for T cell antigen identification, activation, or stimulation. A bead can be of various sizes. In some cases, a bead can be or can be about 3 and 6 micrometers. A bead can be or can be at least about 1, 1.5, 2, 2.5, 3, 3.5, 4, 4.5, 5, 5.5, 6, 6.5, 7, 7.5, 8, 8.5, 9, 9.5, 10 or more micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a poly(lactic-co-glycolic acid) (PLGA) microsphere, a nonspherical particle, a carbon nanotube bundle, a ellipsoid PLGA microparticle, a nanoworms, a fluidic lipid bilayer-containing system, a 2D-supported lipid bilayer (2D-SLBs), a liposome, a RAFTsomes/microdomain liposome, an SLB particle, or any combination thereof. A catch agent can be linked on the bead to capture the secreted polypeptides such as cytokines.

In some cases, an aAPC can be contacted with a CD4+ T cell. In some cases, an aAPC can be contacted with a CD4+ T cell for antigen identification, activation or stimulation. In some cases, an aAPC can activate or expand a CD4+ T cell. For example, an aAPC can be engineered to mimic an antigen processing and presentation pathway of HLA class II-restricted CD4+ T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4+ T cells.

In some cases, an aAPC can be contacted with a CD8+ T cell. In some cases, an aAPC can be contacted with a CD8+ T cell for antigen identification, activation or stimulation. In some cases, an aAPC can activate or expand CD8+ T cells. For example, an aAPC can be engineered to mimic an antigen processing and presentation pathway of HLA class I-restricted CD8+ T cells. For example, engineered K562 cells can be pulsed with an HLA-restricted peptide in order to activate or expand HLA-restricted antigen-specific CD8+ T cells.

In some cases, the use of aAPCs can be combined with exogenously introduced cytokines for T cell activation, expansion, or any combination thereof.

### Catch Agents

The antigen-presenting cell (APC) described herein can comprise a catch agent. In some cases, the catch agent is referred to as a catch reagent in the present disclosure. The catch agent can be soluble or membrane-bound. The catch agent can be associated with the cell surface of the APC. The catch agent can have a specificity to a polypeptide secreted by an activated TCR-expressing cell described herein. For example, the catch agent can be an antibody or fragment thereof having specificity to the polypeptide secreted by the activated TCR-expressing cell.

The catch agent can be membrane-bound (e.g., a protein that can stably associate with the cellular membrane). The catch agent can be a membrane-bound catch agent. For example, the membrane-bound catch agent can be a transmembrane protein. The transmembrane protein can be a receptor expressed by the APC, for example, a cytokine receptor. The receptor can be exogenously expressed by the APC. Various vectors described herein can be used to deliver a nucleic acid molecule encoding a receptor to the APC for expression. The receptor can be a cytokine receptor. The cytokine receptor can be a cytokine receptor targeting any cytokine that secreted by an activated T cell, for example, TNFα tumor necrosis factor alpha (TNFα), TNFβ, interleukin (IL)-1α, IL-1β, IL-2, IL-4, IL-5, IL-6, IL-9, IL-10, IL-12, IL-13, IL-15, IL-17, IL-18, IL-21, IL-22, IL-25, transforming growth factor beta (TGF-β), or interferon (IFN-γ).

The catch agent can be soluble. The catch agent can be a soluble catch agent. The soluble catch agent can be (i) a molecule expressed and/or secreted by the APC or the TCR-expressing cell or (ii) a molecule supplied in the aqueous solution where the APC and the TCR-expressing cell interact. The soluble catch agent may be a molecule expressed and/or secreted by another cell (e.g., the TCR-expressing cell) supplied in the aqueous solution. The soluble catch agent may be contacted with the APC first to be stably associated with the APC before contacting the APC with the TCR-expressing cell. In some cases, the interactions between APCs and TCR-expressing cells are performed within individual compartments, and in such cases, the soluble catch agent may be contacted with the APCs first to be stably associated with the APCs before partitioning the cells into the individual compartments. The soluble catch agent can have specificity to a cell surface protein of the APC and the polypeptide that is secreted by the TCR-expressing cell. The soluble catch agent can bind to both a cell surface protein of the APC and the polypeptide. The cell surface protein can be an endogenous protein or an exogenous protein of the APC. The soluble catch agent can be an antibody or a fragment thereof. The soluble catch agent may not be a transmembrane protein. The soluble catch agent can be directly or indirectly linked to the APC. The catch agent can be a cytokine capturing antibody or fragment thereof.

The catch agent or the soluble catch agent can comprise an antigen binding domain that targets the cell surface protein. The antigen binding domain can be an antibody or a fragment thereof that targets or has specificity to the cell surface protein. Such antibody or a fragment thereof may be obtained from various commercial sources. Non-limiting examples of the cell surface protein include CD11b, CD11c, CD14, CD80, CD86, B7-1, B7-2, CD18, CD29, CD31, CD40, CD43, CD44, CD45, CD54, CD58, CD83, CD86, IFN-γ receptor, IL-2 receptor, ICAM-1, Fcγ receptor, CMRF-44, CMRF-56, DCIR, and DECTIN-1. The soluble catch agent can comprise an additional antigen binding domain that targets a polypeptide secreted by the TCR-expressing cell (e.g., a cytokine). The additional antigen binding domain can be an antibody or a fragment thereof that targets or has specificity to the cytokine. Such antibody or a fragment thereof may be obtained from various commercial sources. The cytokine can be secreted by the TCR-expressing cell (e.g., activated TCR-expressing cell) described herein. The cytokine can be TNFα tumor necrosis factor alpha (TNFα), TNFβ, interleukin (IL)-1α, IL-1β, IL-2, IL-4, IL-5, IL-6, IL-9, IL-10, IL-12, IL-13, IL-15, IL-17, IL-18, IL-21, IL-22, IL-25, transforming growth factor beta (TGF-β), or interferon (IFN-γ). The two antigen binding domains can be linked to produce a catch agent having specificities to both the cell surface protein and the cytokine.

The catch agent can comprise an antibody or fragment thereof. Antibodies can comprise an antigen-binding fragment (Fab) and a fragment crystallizable region(Fc). The Fc region can interact with cell surface receptors which can allow antibodies to activate the immune system. In IgG, IgA and IgD antibody isotypes, the Fc region can comprise two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment may be used for Fc receptor-mediated activity. The N-glycans attached to this site can predominantly be core-fucosylated diantennary structures of the complex type. Examples of antibody fragments include, but are not limited to, (1) a Fab fragment, which is a monovalent fragment consisting of the V_{L}, V_{H}, C_{L}, and C_{H}1 domains, (2) a F(ab′)2 fragment, which is a bivalent fragment comprising two Fab fragments linked by a disulfide bridge at the hinge region, (3) a Fv fragment consisting of the V_{L} and V_{H} domains of a single arm of an antibody, (4) a Fab′ fragment, which results from breaking the disulfide bridge of an F(ab′)₂ fragment using mild reducing conditions, (5) a disulfide-stabilized Fv fragment (dsFv), and (6) a single domain antibody (sdAb) such as VHH domain, which is an antibody single variable domain (V_{H} or V_{L}) polypeptide that specifically binds an antigen.

While the constant domains of the light and heavy chains may not be directly involved in binding of the antibody to an antigen, the constant domains can influence the orientation of the variable domains. The constant domains can also exhibit various effector functions, such as participation in antibody-dependent complement-mediated lysis or antibody-dependent cellular toxicity via interactions with effector molecules and cells.

An antibody can also include chimeric antibodies, humanized antibodies, and recombinant antibodies, human antibodies generated from a transgenic non-human animal, as well as antibodies selected from libraries using enrichment technologies.

An antibody can be a protein that is found in blood or other bodily fluids of vertebrates, which is used by the immune system to identify and neutralize foreign objects, such as bacteria and viruses. An antibody can include monoclonal antibodies, polyclonal antibodies, multispecific antibodies (for example, bispecific antibodies and polyreactive antibodies), and antibody fragments. Thus, an antibody can include, but not be limited to, any specific binding member, immunoglobulin class and/or isotype (e.g., IgG1, IgG2, IgG3, IgG4, IgM, IgA, IgD, IgE and IgM); and biologically relevant fragment or specific binding member thereof, including but not limited to Fab, F(ab′)₂, Fv, and scFv (single chain or related entity). Antibody fragments can be produced by recombinant DNA techniques, or by enzymatic or chemical cleavage of intact antibodies. An antibody other than a “bispecific” or “bifunctional” antibody can be understood to have each of its binding sites identical. A monoclonal antibody can be obtained from a population of substantially homogeneous antibodies, i.e., the individual antibodies comprising the population are identical except for possible naturally occurring mutations that may be present in minor amounts. A polyclonal antibody can be a preparation that includes different antibodies directed against different determinants (epitopes).

The catch agent can be a bispecific antibody (BsAb). The BsAb can be various types of antibodies that have two or more specificities to two or more different targets (e.g., epitopes or antigens). The BsAb can be an antibody produced by a quadroma cell. For example, a method for producing bispecific antibodies can be through the somatic fusion of two hybridoma cell lines, forming a quadroma cell—sometimes referred to as a hybrid hybridoma—capable of secreting whole IgG antibodies with the binding characteristics of the two individual parental hybridomas in a single molecule. The BsAb can be a heterodimeric antibody or a fragment thereof. The BsAb can be a recombinant protein or a bispecific fusion protein.

The BsAb can be a bispecific IgG or a fragment thereof. The bispecific IgG can be a Knobs-in-holes (KIH) BsAb, an orthogonal Fab BsAb, a dual-targeting (DT)-IgG, a SEEDbody, a LUZ-Y BsAb, a charge pair BsAb, a Fab-arm exchange BsAb, κλ-body. The knobs-into-holes (KIH) technique may be suitable for heterodimerization immunoglobulin heavy (H) chains with amino acid changes, which can overcome the mispairing problem. The KIH BsAb can be heterodimeric BsAb, where the Fc domain comprises one or more mutations that result in preferential formation of the heterodimer (e.g., reducing mispairing). The orthogonal Fab BsAb can be a BsAb having two different Fab domains, where mutations can be into Fab interfaces in order to minimize light chain and heavy chain mispairing and get correctly assembled products in one host cell. The DT-IgG can be an antibody that are modified with two targets binding specificity. The SEEDbody can be an antibody produced by the SEED method, which is designed to regulate H-chain heterodimerization so as to generate heterodimer preferentially via sequence exchanging between IgG1 and IgA. The LUZ-Y BsAb can be an antibody produced by introducing a single amino acid mutation into heavy chain and a leucine zipper into the antibody Fc domain C terminus respectively. This structure can play a role in promoting the dimerization process of the heterogenous heavy chains. And it can be easily removed in antibody purification process. The LUZ-Y bispecific antibody can greatly avoid cognate light chains and heavy chains mispairing. The charge pair BsAb can be an antibody where the interface residues can be selectively mutated to construct light chain-heavy chain (LC-HC) pairs to overcome the heavy chain-pairing problem within bispecific antibody production. The Fab-arm exchange BsAb can be an antibody produced by following the steps including, expressing two IgG antibodies separately in mammalian cell lines; introducing a single residue mutation into the third constant (CH3) domain of each antibody; purifying the antibodies through standard protein recovery and purification procedures; and producing the BsAb by recombining of these two antibodies. The κλ-body can be a BsAb having a common heavy chain and two light chains (one kappa and one lambda) with distinct specificities.

The bispecific antibody can be an appended IgG of a fragment thereof. Appended IgGs can be a class of IgG-like bispecific molecules which are produced via connecting the N and/or C terminal of either light and/or heavy chains with additional antigen-binding units, including paired antibody variable domains like Fv or scFv, unpaired VL or VH, or engineered protein scaffolds. The appended IgG can be a dual variable domain IgG, a IgG-scFv, a ScFv4-Ig, a IgG(L, H)-Fv, a IgG-v, a KIH IgG-scFab, a Zybody, or a DIV-IgG. The dual variable domain IgG can be generated from two parental monoclonal antibodies (mAbs) by placing two variable domains from one parental antibody onto the heavy chain and the light chain of another parental antibody, instead of one variable domain. The IgG-scFv BsAb can be a BsAb which is engineered for bispecificity by fusing two scFvs respectively to a monospecific IgG. The specificity of each scFv can be same or different. The ScFv4-Ig can be a BsAb produced by fusing two scFvs to the CL domain and another two different scFvs to the first CH1 domain of the IgG heavy chain, or vice versa. The IgG(L, H)-Fv can be produced by appending VH and VL domains of an antibody to the C-terminal of two distinct heavy chains. The IgG-v BsAb can be produced by appending paired antibody variable domains Fv to the N or C terminus of the heavy or the light chain of an antibody, resulting in multi specific molecules with two binding sites for each antigen. The KIH IgG-scFab can be produced by fusing a scFab domain to the C-termini of IgG heavy chain modified by KIH method. The Zybody can be produced by fusing modular recognition domains to N- and C-termini of both the heavy and the light chains of a full-length mAb. The DVI-IgG can be a BsAb comprising two additional binding domains besides the two binding domains of the original bispecific antibody.

The BsAb can be a bispecific antibody fragment. The bispecific antibody fragment can be a bi-single domain antibody (sdAb), a dual-affinity re-targeting antibody (DART), a tandem diabody (TandAb), a tandem scFv, a tandem scFv-Fc BsAb, diabody, a mini-antibody, a minibody, a scFv fragment BsAb, a Fab fragment BsAb, a scFv-Fab fragment BsAb, or a bispecific intrabody. The bi-sdAb can be a BsAb having a first VHH domain linked to a second VHH domain. The DART can be a BsAb having two engineered Fv fragments which have their own VH exchanged with the other one. The TandAb can be a BsAb produced by artificial vectors encoding tandem two VLs/VHs pairs from two distinct Fv. The tandem scFv-Fc BsAb can be a BsAb produced by expression vectors having genes coding each single-chain variable domain tandemly arrayed to a human Fc gene. The diabody can comprise two chains expressed separately, each of which consists of a VH from one antibody and a VL from another antibody. The mini-antibody can be an artificial immunoglobin without CH1 domain. Each single chain of a mini-antibody can be expressed separately in prokaryotic or eukaryotic expression system and linked to another single chain with distinct specificity via chemical modification or KIH method to get bispecific antibody. The minibody can be comprise a pair of single-chain Fv fragments which are linked via CH3 domains, and Fvs with distinct specificity, which paired to the former part through heterodimerization process. To promote the heterodimerization efficiency, single-residue mutations can be introduced into each CH3 domains (e.g., KIH method). The scFv fragment BsAb can comprise two different scFv domains linked to the two chains of a Fc domain or a CH3 domain. The scFv fragment BsAb can also comprise a first scFv linked to a CH and a second scFv linked to a CL. The Fab fragment BsAb can be a F(ab′)₂ fragment having a first Fab domain and a second Fab domain, where the two Fab domains have different specificities. The scFv-Fab BsAb can comprise a scFv fragment linked to a heavy chain or a Fab light chain of another antibody. Fab-Fc fragment and scFv-Fc fragment with distinct specificity can also be paired through the interaction between Fc domains, which can form bispecific Fab-scFv-Fc antibody. The bispecific intrabody can comprise two identical immunoglobin chains paired via its recombinant Fc domains. Two pairs of scFvs with distinct specificity can be linked to each end of the Fc domain respectively.

The bispecific antibody can be a bispecific fusion protein. The bispecific fusion protein can be a bispecific antibody-HSA fusion protein or a tandem scFv-toxin bispecific fusion protein.

The bispecific antibody can be a bispecific antibody conjugate. The bispecific antibody conjugate can be a IgG-IgG BsAb, a Cov-X-body, or a scFv-(chemical linker)-scFv.

The bispecific antibody can comprise a first antigen binding domain targeting the cell surface protein of the APC and a second antigen binding domain targeting the secreted polypeptide. The first antigen binding domain and the second antigen binding domain can be linked by a linker. The linker can be a chemical linker or a peptide linker. The first antigen binding domain or the second antigen binding domain can be a full-length antibody or a fragment thereof. For example, the first antigen binding domain or the second antigen binding domain can be an immunoglobulin (e.g., IgG, IgA or IgM), a scFv or a sdAb. For example, the bispecific antibody can comprise two different monoclonal antibodies crossed linked through Fc domains.

In some embodiments, the catch agent can be associated with the APC indirectly. For example, the catch agent can be associated with (e.g., linked to, attached to, bound to, crosslinked to, or immobilized on) a solid support described herein. The solid support can also be associated with (e.g., linked to, attached to, bound to, or crosslinked to) the APC. The association can be a covalent or non-covalent interaction. In some cases, the solid support can be used to entrap or embed the APC as described herein. Various methods can be used to associate the catch agent or the APC to the solid support. For example, the catch agent can be associated with the solid support through the interaction between an affinity binding pair. Example affinity binding pairs include any haptenic or antigenic compound in combination with a corresponding antibody or binding portion or fragment thereof (e.g., digoxigenin and anti-digoxigenin; mouse immunoglobulin and goat anti-mouse immunoglobulin) and nonimmunological binding pairs (e.g., biotin-avidin, biotin-streptavidin, biotin-neutravidin, hormone (e.g., thyroxine and cortisol-hormone binding protein), receptor-receptor agonist, receptor-receptor antagonist (e.g., acetylcholine receptor-acetylcholine or an analog thereof), IgG-protein A, IgG-protein G, IgG-synthesized protein AG, lectin-carbohydrate, enzyme-enzyme cofactor, enzyme-enzyme inhibitor, and complementary oligonucleotide pairs capable of forming nucleic acid duplexes), and the like. For example, the solid support can be functionalized with streptavidin, and biotinylated catch agent (e.g., antibody) specific for the polypeptide (e.g., cytokine) secreted by the TCR-expressing cell can be bound to the streptavidin through biotin-streptavidin interaction. In some cases, the catch agent can be associated with the solid support by a covalent bond such as a crosslinker. Non-limiting examples of crosslinkers can include, but are not limited to, amine-to-amine crosslinkers (e.g., but are not limited to the ones based on NETS-ester and/or imidoester reactive groups), amine-to-sulfhydryl crosslinkers, carboxyl-to-amine crosslinkers (e.g., but are not limited to, carbodiimide crosslinking agents such as DCC, and/or EDC (EDAC); and/or N-hydroxysuccinimide (NETS)), photoreactive crosslinkers (e.g., but not limited to, aryl azide, diazirine and any art-recognized photoreactive (light-activated) chemical crosslinking reagents), sulfhydryl-to-carbohydrate crosslinkers (e.g., but are not limited to the ones based on maleimide and/or hydrazide reactive groups), sulfhydryl- to hydroxyl crosslinkers (e.g., but are not limited to the ones based on maleimide and/or isocyanate reactive groups), sulfhydryl-to-sulfhydryl crosslinkers (e.g., but are not limited to, maleimide and/or pyridyldithiol reactive groups), sulfo-SMCC crosslinkers, sulfo-SBED biotin label transfer reagents, sulfhydryl-based biotin label transfer reagents, photoreactive amino acids (e.g., but are not limited to diazirine analogs of leucine and/or methionine), NHS-azide Staudinger ligation reagents (e.g., but are not limited to, activated azido compounds), NHS-phosphine Staudinger ligation reagents (e.g., but are not limited to, activated phosphine compounds), and any combinations thereof. The covalent bond can be formed by reactive groups. Examples of suitable reactive groups include electrophiles or nucleophiles that can form a covalent linkage by reaction with a corresponding nucleophile or electrophile, respectively, on the substrate of interest. Non-limiting examples of suitable electrophilic reactive groups may include, for example, esters including activated esters (for example, succinimidyl esters), amides, acrylamides, acyl azides, acyl halides, acyl nitriles, aldehydes, ketones, alkyl halides, alkyl sulfonates, anhydrides, aryl halides, aziridines, boronates, carbodiimides, diazoalkanes, epoxides, haloacetamides, haloplatinates, halotriazines, imido esters, isocyanates, isothiocyanates, maleimides, phosphoramidites, silyl halides, sulfonate esters, sulfonyl halides, and the like. Non-limiting examples of suitable nucleophilic reactive groups may include, for example, amines, anilines, thiols, alcohols, phenols, hydrazine, hydroxylamines, carboxylic acids, glycols, heterocycles, and the like. For example, the solid support can be functionalized with an amine reactive group (e.g., aldehyde), which can react with an amine group on the catch agent (e.g., amide containing antibody).

The solid support such as a microbead may be modified with an antibody which can bind a cell surface protein on the APC, which can lead to physical association between the APC and the solid support. The antibody can be covalently or non-covalently linked to the solid support.

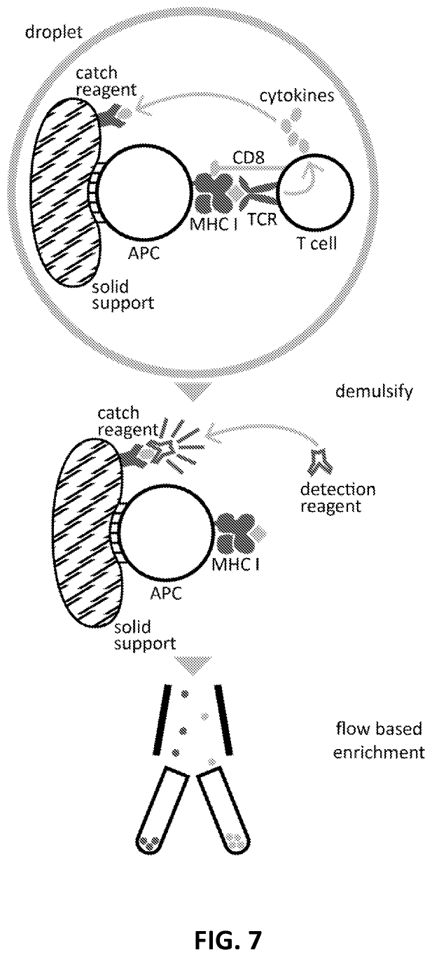

The solid support may be selected along with the APC to isolate the APC that is recognized by the TCR-expressing cell (FIG. 7). FIG. 7 shows an example of the methods described herein. In this example, the catch agent can be associated with the APC indirectly within a compartment (e.g., a droplet). The catch agent can be associated with a solid support, which can be in turn associated with the APC. The catch agent can bind to the polypeptide secreted by the TCR-expressing cell after recognizing the antigen presented by the APC. The solid support with the catch agent and APC associated thereon can be released from the compartment, and the secreted polypeptide bound by the catch agent can be bound by a detection agent. The complex comprising the solid support, the catch agent, the secreted polypeptide, the APC, and the detection agent can be selected, for example, by follow-based cell sorting, to select the APC that is recognized by the TCR-expressing cell.

In some embodiments, the catch agent can be associated with the cell surface of the APC directly (e.g., without the solid support). The catch agent can be covalently or non-covalently linked to the cell surface of the APC directly. The catch agent can be an antibody or fragment thereof. The antibody or fragment thereof can be a monospecific antibody. For example, the antibody or fragment thereof can be linked to the cell surface of the APC via a chemical linker. In some cases, the antibody or fragment thereof can comprise or be functionalized with a reactive group, and the cell surface of the APC can comprise or be functionalized with another reaction group which can form a chemical bond with the reactive group on the antibody or fragment thereof. The reactive group can be any reactive group described in the present disclosure. In some cases, the reactive group can comprise or can be a conjugation handle, and the association of the antibody or fragment thereof and the APC can be formed via click chemistry. For example, the antibody or fragment thereof can comprise or be functionalized with a first conjugation handle such as transcyclooctene (TCO), and the cell surface of the APC can comprise or be functionalized with a second conjugation handle such as tetrazine, or vice versa. Additional example pairs of conjugation handles include, but are not limited to, azide and alkyne, and azide and dibenzocyclooctyne (DBCO). Functionalization can be achieved by reacting the conjugation handle with a primary amine group. Antibodies or cell surface can comprise such primary amines for functionalization. In some cases, the association of the catch agent and the APC can be non-covalent. For example, the catch agent can be associated with the cell surface of the APC through the interaction between an affinity binding pair. The affinity binding pair can be any pair such as those described herein. In some cases, the cell surface of the APC can comprise or be functionalized with streptavidin, and biotinylated catch agent (e.g., antibody or fragment thereof) can be bound to the streptavidin through biotin-streptavidin interaction. Functionalization of the cell surface with streptavidin can be achieved by various methods, for example, by biotinylating cell surface and then coating the surface with streptavidin. In some cases, the cell surface of the APC can comprise or be linked to a first nucleic acid strand, the catch agent can comprise or be linked to a second nucleic acid strand complementary to the first nucleic acid strand, and the association between the APC and the catch agent can be formed via hybridization.

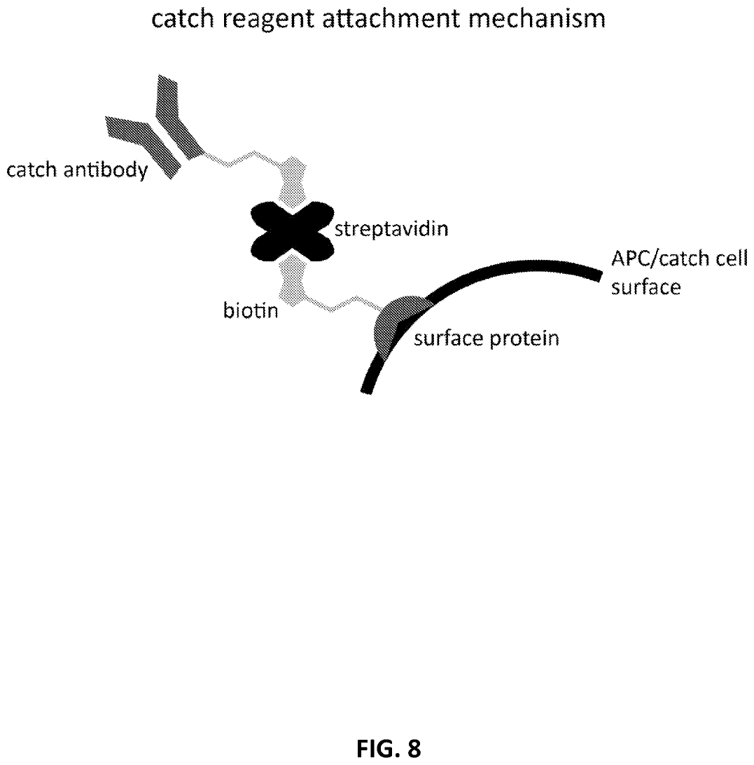

FIG. 8 shows an example of the methods described herein. In this example, a catch antibody, which can specifically bind to the cytokine secreted from an activated T cell, is directly linked to the surface of the APC without a solid support as in FIG. 7. The linkage between the catch antibody and the APC can be the interaction between streptavidin and biotin. First, a surface protein of the APC can be biotinylated. Next, the surface of the APC can be coated with streptavidin. Next, a biotin-conjugated catch antibody can be contacted with the streptavidin-coated APC and linked to the APC through the biotin-streptavidin interaction.

### Detection Agents

The compositions or methods provided herein can comprise a detection agent. In some cases, the detection agent is referred to as a detection reagent. The detection agent can be used to detect the APCs comprising antigens recognized by a TCR. For example, the method provided herein can comprise contacting the polypeptide (e.g., a cytokine) bound to the catch agent associated with the APC with a detection agent, wherein the detection agent binds to the polypeptide secreted by the TCR-expressing cell.

The detection agent can be an antibody or fragment thereof. The detection agent can have specificity to the polypeptide. The detection agent and the catch agent can bind to different epitopes of the polypeptide. The detection agent and the catch agent may be both antibodies or fragments thereof. The detection agent and the catch agent may be both antibodies or fragments thereof that target a cytokine. Many pairs of antibodies that used in sandwich ELISA assay for a cytokine can be used as the catch agent and the detection agent. Both of the catch agent and the detection agent can be monoclonal or polyclonal antibodies. In some cases, if one antibody is a polyclonal antibody and the other is a monoclonal antibody, the capture agent may be the monoclonal antibody. In some cases, if one antibody is a polyclonal antibody and the other is a monoclonal antibody, the detection agent may be the monoclonal antibody. For example, when the cytokine is IFN-γ, the catch agent can be clone AbD00676, and the detection agent can be clone AbD02503, or vice versa. For another example, when the cytokine is IL-2, the catch agent can be clone MQ1-17H12, and the detection agent can be clone Poly5176.

The detection agent can comprise a signal. The signal can be a detectable label. The detectable label can be a fluorescent label.

Examples of detectable label include, but are not limited to, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, phycoerythrin (e.g., R-phycoerythrin or PE), Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2 (and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores. In some cases, the detection agent is a phycoerythrin (PE)-conjugated antibody. The APCs containing the secreted cytokines can be enriched using magnetic selection with anti-PE microbeads.

The detection agent can comprise an affinity tag (e.g., a biotin or derivative thereof). The affinity tag can be captured by its corresponding tag binder (e.g., streptavidin or derivative thereof) in order to select or enrich the APCs bound with the detection agent via affinity chromatography. For example, the detection agent may be an antibody or fragment thereof and can be conjugated with the affinity tag. Non-limiting examples of affinity tags and the corresponding tag binders include digoxin and anti-digoxin antibody, fluorescein and anti-fluorescein antibody, His tag and metal ion such as nickle, FLAG tag and anti-FLAG antibody, Rho1D4 tag and anti-Rho1D4 antibody, Strep tag and and engineered streptavidin, GST tag and gluthathione.

When the methods described herein are performed in a plurality of compartments, the detection agent can be contacted with the secreted polypeptide after releasing the cells from the plurality of compartments. Alternatively, the detection agent can be co-partitioned into the plurality of compartments, and then after the detection agent bound to the polypeptide captured by the catch agent associated with the APC, the cells can be released from the plurality of compartments.

### Methods of Antigen Identification

The present disclosure provides compositions and methods to identify antigen-presenting cells (APCs) or the associated antigens that can be recognized by TCRs in a plurality of APCs, each presenting a different candidate antigen. The polypeptides (e.g., cytokines) secreted by the activated TCR-expressing cells may be diffusion-restricted such that the secreted polypeptides can be captured by the APC presenting the antigen recognized by the TCR, but not other APCs presenting unrelated antigens.

An individual APC presenting a candidate antigen can be partitioned with one or more TCR-expressing cells within a single compartment such that the polypeptides (e.g., cytokines) secreted by the activated TCR-expressing cell can be captured by the APC without diffusing away or be captured by another APC presenting another candidate antigen that is not recognized by the one or more TCR-expressing cells. Before partitioning, the APCs and the TCR-expressing cells can be mixed. The number of TCR-expressing cells may be at least about 2, 3, 4, 5, 6, 7, 8, 9, 10, 11, 12, 13, 14, 15, 16, 17, 18, 19, 20 or more times more than the number of APCs in the mixture. In such cases, when partitioning, an individual compartment may comprise one APC and one or more TCR-expressing cells. The TCR-expressing cells can be non-activated cells prior to being partitioned into individual compartments. The APCs may not have been contacted with any T cells prior to being partitioned into individual compartments.

The compartment can be a tube, a well, a microwell, or a water-in-oil droplet. In some cases, the compartment can be a water-in-oil droplet in an emulsion. Using water-in-oil droplets can offer ultra-high throughput since millions or more of such droplets can be created in a few minutes to hours. Generation of water-in-oil droplets can be achieved by vortexing or using microfluidic chips such as a flow-focusing microfluidics chip. In some embodiments, the emulsion is formed passively using a microfluidics device. These methods can involve squeezing, dripping, jetting, tip-streaming, tip-multi-breaking, or similar. Passive microfluidic droplet generation can be modulated to control the particle number, size, and diameter by altering the competing forces of two different fluids. These forces can be capillary, viscosity, and/or inertial forces upon the mixing of two solutions. In some embodiments, the emulsion is formed by active control of a microfluidics chip. In active control, droplet generation can be manipulated via external force application, such as electric, magnetic, or centripetal forces. A popular method for controlling active manipulation of droplets in a microfluidic chip is to modify intrinsic forces by tuning fluid velocities of two mixing solutions, such as oil and water.

It should be understood that the method may not be limited to be performed in individual compartments. For example, the method may be performed on or within a solid support, where the APCs and TCR-expressing cells can be deposited on or associated with the solid support. An APC presenting a candidate antigen and a TCR-expressing cell can be closely located on the solid support to allow interactions, but are in a certain distance (e.g., at least about 100, 150, 200, 250, 300, 350, 400, 450, 500, 550, 600, 650, 700, 750, 800, 850, 900, 950, 1,000 or more μm) away from other APC and TCR-expressing cell pairs such that the polypeptide secreted by an activated TCR-expressing cell may not diffuse away and the APC presenting the antigen antigen recognized by the TCR can capture the polypeptide. For example, the solid support can be beads, in which case the beads can be immersed in a continuous volume of aqueous solution so that the reactants (e.g., cells or molecules) associated with the beads can access the reactants or reagents in the aqueous solution. For example, the solid support can be the surface of solid microwells patterned on a larger solid surface. In this case the entire solid surface can be immersed in a continuous volume of aqueous solution so that the reactants on the microwells can access the reactants in the aqueous solution. For another example, the solid support can be a well or a cell culture flask. The cells may be in suspension or adherent. The cells may be placed within the well or the cell culture flask at a low density such that only APCs in contact with the activated T cells bind to the secreted cytokines. In some cases, the APCs are in suspense, and the cells may be placed in the well or the cell culture flask at a density of at most about 10⁷, 5×10⁶, 2.5 ×10⁶, 10⁶, 5 ×10⁵, 2.5 ×10⁵, 10⁵, 5 ×10⁴, 2.5 ×10⁴, 10⁴ or less cells per milliliter (cells/mL). In some cases, the APCs are in suspense, and the cells may be placed in the well or the cell culture flask at a density of about 2.5×10⁵ cells/mL. In some cases, the APCs are adherent, and the cells may be placed in the well or the cell culture flask at a density of at most about 10⁶, 5 ×10⁵, 2.5 ×10⁵, 10⁵, 5 ×10⁴, 2.5 ×10⁴, 10⁴, 5 ×10³, 2.5 ×10³, 10³ or less cells per milliliter (cells/mL). In some cases, the APCs are adherent, and the cells may be placed in the well or the cell culture flask at a density of about 10⁴ to about 10⁵ cells/mL. When co-culturing the APCs and the TCR-expressing cells, the ratio of TCR-expressing cell to the APC can be from 1:10 to 10:1. In some cases, the ratio of TCR-expressing cell to the APC can be at most about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, or less.

For another example, the solid support can be a gel matrix (e.g., agar, agarose or polyacrylamide gel), wherein the reactants can be embedded within the gel matrix such that the polypeptide secreted by the TCR-expressing cell may not diffuse away. In other words, the polypeptide can be diffusion-restricted. In this case, the APCs and TCR-expressing cells may be contacted within an aqueous solution containing a plurality of polymerizable or gellable polymers and/or monomers for interaction. A gel matrix can be formed by polymerizing or gelling the polymerizable or gellable polymers and/or monomers such that the APCs and TCR-expressing cells along with molecules secreted from the cells can be diffusion-restricted. The polymerizable or gellable polymers can comprise polysaccharides, polyacrylamides, polyacrylic acids, polyethylene glycols, polyvinyl alcohols, polymethacrylamides, or any combinations thereof. Examples of the polysaccharides include, but are not limited to, agarose, hyaluronic acids, carboxymethycellose, chitosan, alginate, or any combinations thereof. The polymerizable or gellable monomers can comprise acrylic acids, acrylamides, methacrylamides, methacrylic acids, or any combinations thereof. The polymerizing or gelling can be triggered by temperatures or initiators. The polymerization initiator can be a photo-initiator. For example, the polymerization initiator can be ammonium persulfate (APS), N,N,N′,N′-tetramethylethane-1,2-diamine (TEMED), Lithium- and magnesium phenyl-2,4,6-trimethylbenzoylphosphinates (TMPPL and TMPPM), sodium 4-[2-(4-morpholino)benzoyl-2-dimethylamino]-butylbenzenesulfonate (MBS), methylated-β-cyclodextrin (MβCD), 2,2-dimethoxy-2-phenyl acetophenone (DMPA), or any combinations thereof.

Solid supports can be flat or planar, or can have substantially different conformations. For example, the solid support can exist as particles, beads, strands, precipitates, gels, sol-gels, sheets, tubing, spheres, containers, capillaries, pads, slices, films, plates, dipsticks, slides, etc. Magnetic beads or particles, such as magnetic latex beads and iron oxide particles, are examples of solid substrates.

Example materials that can form solid supports include glasses or other ceramics, plastics, polymers, metals, metalloids, alloys, composites, organics, etc. For instance, the solid supports can comprise a material selected from a group consisting of: silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys can also be options for use as solid supports. In addition, many ceramics and polymers can also be used as solid supports. Polymers which can be used as solid supports include, but are not limited to, the following: polystyrene; poly(tetra)-fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyatkenesulfone (PAS); polypropylene; polyethylene; polyhydroxyethylmethacrylate (HEMA); polydimethyl-siloxane; polyacrylamide; polyimide; and block-copolymers. The solid support can be composed of a single material (e.g., glass), mixtures of materials (e.g., co-polymers) or multiple layers of different material (e.g., metal coated with a monolayer of small molecules, glass coated with a BSA, etc.).

The configuration of a solid support can be any appropriate form, e.g., can comprise beads, spheres, particles, granules, a gel, a sol-gel, a self-assembled monolayer (SAM) or a surface (which can be flat, or can have shaped features). The solid support can include semisolid supports. Surfaces of the solid support can be planar, substantially planar, or non-planar. Solid supports can be porous or non-porous, and can have swelling or non-swelling characteristics. A solid support can be configured in the form of a well, depression or other container, vessel, feature or location. A plurality of solid supports can be configured in an array at various locations, addressable for robotic delivery of reagents, or by detection methods including scanning by laser or other illumination and CCD, confocal or deflective light gathering.

In some embodiments, the solid support is in the form of a bead (synonymous with particle). A bead can be made of any substrate material, including biological, non-biological, organic, inorganic, polymer, metal, or a combination of any of these. The surface or interior of the bead can be chemically modified and subject to any type of treatment or coatings, e.g., coatings that contain reactive groups that permit binding interactions with the tool molecules.

A solid support may be a hydrogel particle. Hydrogel can be made into hydrogel particles using existing methods. For example, the sol state or the precursors of the hydrogel can be made into water-in-oil emulsions. The aqueous droplets can be turned into gel state (e.g., by polymerization of the precursors, or by lowering the temperature for thermo-reversible hydrogels such as agarose) to yield ‘hydrogel-in-oil’ emulsions. Polymerization of the precursors can be triggered by light or by adding initiator or accelerator (e.g., TEMED) in the oil phase. This emulsion can be demulsified to yield hydrogel particles suspended in aqueous solution.

In some embodiments, a solid support may be within a compartment comprising the APC and TCR-expressing cell. For example, a particle such as a microbead (as an example of the solid support) may be within a water-in-oil droplet along with the APC and TCR-expressing cell (FIG. 7). The solid support may be manufactured before the generation of the compartment and partitioned into the compartment along with the APC and TCR-expressing cell. The solid support may ‘template’ the formation of the compartment. For example, when an aqueous solution containing particles (e.g., hydrogel particles) is mixed with oil and agitated, a droplet may form around the particle. A volume of the liquid (e.g., at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, or at least about 1000% of the particle volume) may be present around the particle in the droplet and this small volume of liquid may contain APC or the TCR-expressing cell. The particle may be spherical or non-spherical. If the particle is non-spherical, e.g., if the particle has a cavity, a more controllable volume of liquid (e.g., at least about 10%, at least about 20%, at least about 30%, at least about 40%, at least about 50%, at least about 60%, at least about 70%, at least about 80%, or at least about 90% of the particle volume) may be trapped in the droplet along with the particle.

Alternatively, the solid support may be formed in the compartment after the compartment is generated, for example, if the solid support is a hydrogel particle described herein. For example, the precursors (e.g., polymerizable or gellable polymers or monomers) of the hydrogel particle along with the APC and the TCR-expressing cell can be co-partitioned into a compartment, and subsequently, the precursors can be polymerized or crosslinked to form a hydrogel particle within the compartment. In this case, the APC and the TCR-expressing cell can be entrapped or embedded within the gel matrix within the compartment. The polymers used to form the hydrogel particles can be polysaccharides, polyacrylamides, or a combination thereof. The polysaccharides can be agarose, hyaluronic acids, carboxymethycellose, chitosan, starch, dextran, or alginate. The monomers used to form the hydrogel particles can be acrylamide or methacrylamide monomers. The polymerized or gelled polymers and/or monomers can comprise a mixture of agarose and polyacrylamides. The polymerized or gelled plurality of polymers and/or monomers can be cross-linked. In some cases, the polymerizable or gellable polymers and/or monomers can be polymerized or gelled by using an imitator. The initiator can be a UV light or a chemical. In some cases, the plurality of polymerizable or gellable polymers and/or monomers can be polymerized or gelled by reducing temperature of the vessel. For example, agarose particle can be formed by reducing the temperature of the agarose.

The solid support in the compartment may be associated to (e.g., linked to, crosslinked to, immobilized to) or entrapping the APC. For example, a solid support such as a microbead may be covalently modified with an antibody which can bind a cell surface protein on the APC, which can lead to physical association between the APC and the solid support. Non-limiting examples of the cell surface protein include CD11b, CD11c, CD14, CD80, CD86, B7-1, B7-2, CD18, CD29, CD31, CD40, CD43, CD44, CD45, CD54, CD58, CD83, CD86, IFN-γ receptor, IL-2 receptor, ICAM-1, Fcγ receptor, CMRF-44, CMRF-56, DCIR, and DECTIN-1. Antibodies or fragment hereof targeting those cell surface proteins can be available from various commercial sources. If the solid support is formed after the compartments are generated, e.g., if the solid support is formed by turning the precursors of hydrogel in the water-in-oil droplet into a hydrogel bead, the solid support (e.g., in this case, the hydrogel bead) may entrap the APC and thus physically associate with the APC.

In some aspects, the present disclosure provides a method of identifying an APC comprising an antigen recognized by a TCR. The method can comprise providing a plurality of APCs, each comprising a different antigen complexed with a major histocompatibility complex (MEW) molecule, and a plurality of TCR-expressing cells comprising a TCR. Next, the plurality of APCs and the plurality of TCR-expressing cells can be partitioned into a plurality of compartments. A compartment of the plurality of compartments can comprise (i) an APC of the plurality of APCs and (ii) at least one TCR-expressing cell of the plurality of TCR-expressing cells. In some cases, each compartment of the plurality of compartments can comprise (i) an APC of the plurality of APCs and (ii) at least one TCR-expressing cell of the plurality of TCR-expressing cells. The compartment or each compartment of the plurality of compartments can comprise a single APC. Next, an antigen complexed with an MEW molecule of the APC can bind to the TCR of the at least one TCR-expressing cell within the compartment. The at least one TCR-expressing cell can secrete a polypeptide (e.g., a cytokine) upon binding with the APC. The polypeptide that is secreted can bind to a catch agent associated with the APC. Next, the polypeptide bound to the catch agent associated with the APC can be detected or selected (e.g., by flow cytometry) to identify the APC comprising the antigen recognized by the TCR.

The catch agent can be a membrane-bound catch agent. The membrane-bound catch agent can be a protein stably attached to a cellular membrane. The membrane-bound catch agent can be a transmembrane protein. For example, the transmembrane protein is a receptor (e.g., a cytokine receptor) expressed by the APC. The receptor can be exogenously expressed by the APC.

The catch agent may not be membrane-bound. The catch agent can be a soluble catch agent. The soluble catch agent can be a molecule expressed and/or secreted by the APC, the TCR-expressing cell, or another cell supplied (e.g., co-partitioned) within the compartment. For example, the APC, the TCR-expressing cell or another cell within the compartment can be engineered to express or secrete the soluble catch agent. The soluble catch agent can be a molecule supplied within the compartment. The soluble catch agent can have specificity to a cell surface protein of the APC and the polypeptide that is secreted by the TCR-expressing cell. The soluble catch agent can bind to both a cell surface protein of the APC and the polypeptide. The cell surface protein can be an endogenous protein or an exogenous protein of the APC. The soluble catch agent can be an antibody or a fragment thereof. The antibody can be a bispecific antibody (BsAb) or a multi-specific antibody. The BsAb can be an antibody produced by a quadroma cell. The BsAb can be a heterodimeric antibody or a fragment thereof. The BsAb can be a recombinant protein or a bispecific fusion protein. The bispecific antibody can comprise a first antigen binding domain targeting the cell surface protein of the APC and a second antigen binding domain targeting the polypeptide that is secreted. The first antigen binding domain and the second antigen binding domain can be linked by a linker. The linker can be a peptide linker or a chemical linker (e.g., polyethylene glycol (PEG), SPDP (succinimidyl-3(2-pyridylthiol) propionate), or (2-iminothiolane)/Sulpho-SMCC (sulpho-[succinimidyl-4-(N-maleimidomethyl)-4-cyclohexane-1-carboxylate])). The first antigen binding domain or the second antigen binding domain can be a IgG, scFv or sdAb.

The cell surface protein can be CD11b, CD11c, CD14, CD80, CD86, B7-1, B7-2, CD18, CD29, CD31, CD40, CD43, CD44, CD45, CD54, CD58, CD83, CD86, IFN-γ receptor, IL-2 receptor, ICAM-1, Fcγ receptor, CMRF-44, CMRF-56, DCIR, or DECTIN-1. The polypeptide can be a cytokine or a fragment thereof (e.g., a functional fragment). The cytokine can be TNFα tumor necrosis factor alpha (TNFα), TNFβ, interleukin (IL)-1α, IL-1β, IL-2, IL-4, IL-5, IL-6, IL-9, IL-10, IL-12, IL-13, IL-15, IL-17, IL-18, IL-21, IL-22, IL-25, transforming growth factor beta (TGF-β), or interferon (IFN-γ). For example, the cell surface protein can be CD45, and the cytokine can be selected from the group consisting of IL-2, IL-4, IL-10, IL-12, IFN-γ and TNFβ. In some cases, the cytokine can be IL-2. For another example, the cell surface protein can be CD11b, and the cytokine can be selected from the group consisting of IL-2, IL-4, IL-10, IL-12, IFN-γ, TNFα and TNFβ. In some cases, the cytokine can be IFN-γ.

The method provided herein can comprise detecting the polypeptide (e.g., cytokine) bound to the catch agent associated with the APC. The detecting method can comprise contacting the polypeptide bound to the catch agent associated with the APC with a detection agent. The detection agent can bind to the polypeptide. The detection agent can have specificity to the polypeptide. The detection agent can be an antibody or fragment thereof. The detection agent can comprise a signal. The signal can be a detectable label. The detectable label can be a fluorescent label. The methods provided herein can further comprise releasing the plurality of APCs from the plurality of compartments prior to detecting or subsequent to binding of the APC and the TCR-expressing cell. The APC bound with the polypeptide can be released from the compartment. The plurality of compartments can be a plurality of droplets, and the releasing can comprise demulsifying the plurality of droplets.

The methods provided herein can comprise selecting the APC comprising the antigen recognized by the TCR. The selecting can comprise contacting the polypeptide (e.g., cytokine) bound to the catch agent associated with the APC with a detection agent, wherein the detection agent binds to the polypeptide. The detection agent can comprise a signal or an affinity tag. The selecting can comprise selecting the APC comprising the antigen recognized by the TCR based on the signal or the affinity tag of the detection agent. For example, the selecting can comprise sorting the plurality of APCs by flow cytometry (e.g., fluorescence-activated cell sorting or FACS) based on the signal from the detection agent. The APC comprising the antigen recognized by the TCR can be selected (e.g., sorted out or enriched). For another example, the selecting can comprise capturing the affinity tag of the detection agent (e.g., using tag binder to capture the affinity tag). The methods provided herein can further comprise releasing the plurality of APCs from the plurality of compartments prior to selecting or subsequent to binding of the APC and the TCR-expressing cell.

The APC can comprise a nucleic acid molecule encoding the antigen. For example, the APC may be pulsed with or engineered to express a defined antigen, a set of defined antigens or a set of undefined antigens (such as tumor lysate or total tumor mRNA). The nucleic acid molecule encoding an antigen can be delivered into the APC by a vector. Various vectors described herein can be used. For example, the vector can be a viral vector, a non-viral vector or a self-amplifying RNA. The nucleic acid molecule encoding an antigen can comprise a full coding sequence of a gene. The gene can be any gene in the genome of an organism. The nucleic acid molecule encoding an antigen can comprise a partial sequence or a fragment of a gene. The methods can further comprise sequencing the nucleic acid or a derivative thereof to identify the antigen. Various sequencing methods can be used to sequence the nucleic acid or a derivative thereof. For example, the APC(s) selected by the methods provided herein can be lysed to release nucleic acids. The released nucleic acids can be processed (e.g., amplified and/or tagged with sequencing adaptors) to generate a sequencing library and subject to sequencing. For example, primers can be designed to target backbone sequences of the vector that flank the nucleic acid sequence encoding the antigen such that the nucleic acid molecule encoding the antigen can be amplified. The various sequencing methods include, but are not limited to, Sanger sequencing, high-throughput sequencing, sequencing-by-synthesis, single-molecule sequencing, sequencing-by-ligation, RNA-Seq, Next generation sequencing (NGS), Digital Gene Expression, Clonal Single MicroArray, shotgun sequencing, Maxim-Gilbert sequencing, or massively-parallel sequencing.

The plurality of APCs can be various APCs described herein. For example, the plurality of APCs can comprise artificial APCs (aAPCs). The aAPCs can comprise cells engineered to express an MHC molecule. The cells engineered to express an MHC molecule can be a cell line or cells isolated from a subject. The cell line can be K562. The cells isolated from a subject can be tumor cells. The plurality of APCs can comprise professional APCs or non-professional APCs. The professional APCs can comprise dendritic cells, macrophages, or B cells. The non-professional APCs can comprise fibroblasts, thymic epithelial cells, thyroid epithelial cells, glial cells, pancreatic beta cells, or vascular endothelial cells. The plurality of TCR-expressing cells can comprise T cells. The T cells can comprise a CD4+ or CD8+ T cell. The T cells can comprise a cytotoxic T cell, an ancillary T cell, a natural killer T cell, an alpha beta T cell, a gamma delta T cell, a regulatory T cell or a memory T cell. The T cells are a cell line.

The individual compartment of the plurality of compartments can comprise a single APC. The individual compartment can comprise a single APC and one or more TCR-expressing cells. In some cases, the individual compartment can comprise a single APC and a single TCR-expressing cell. The plurality of compartments can comprise at least about 5, 10, 20, 30, 40, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more compartments.

In some aspects, the present disclosure provides a method of identifying an antigen-presenting cell (APC) comprising an antigen recognized by a T-cell receptor (TCR). The antigen can be presented by the APC. The antigen can be in complex with an WIC molecule. The method can comprise providing a plurality of antigen-presenting cells (APCs) comprising a first APC and a second APC and a plurality of TCR-expressing cells comprising the TCR. The first APC can comprise a first antigen that can be recognized by the TCR and the second APC can comprise a second antigen that may not be recognized by the TCR. Next, the first APC and the second APC can be contacted with the plurality of TCR-expressing cells. A TCR-expressing cell of the plurality can bind to the first antigen of the first APC and secrete a polypeptide (e.g., a cytokine) upon binding. The polypeptide that is secreted can bind to a first catch agent associated with the first APC. The polypeptide can be diffusion-restricted such that it may not bind to a second catch agent associated with the second APC. The polypeptide bound to the first catch agent associated with the first APC can then be detected, thereby identifying the first APC as the APC comprising the antigen recognized by the TCR. For example, the plurality of APCs and the TCR-expressing cells can be contacted within an aqueous solution containing a plurality of polymerizable or gellable polymers and/or monomers for interaction. The number of TCR-expressing cells may be at least about 2, 3, 4, 5, 6, 7, 8, 9, 10, 11, 12, 13, 14, 15, 16, 17, 18, 19, 20 or more times more than the number of APCs. A gel matrix can be formed by polymerizing or gelling the polymerizable or gellable polymers and/or monomers such that the APCs and TCR-expressing cells along with molecules (e.g., the cytokines) secreted from the cells can be diffusion-restricted. The gel matrix can be formed after contacting the plurality of APCs and the TCR-expressing in the aqueous solution for at least about 5, 10, 15, 20, 25, 30, 35 or more minutes. The second APC having a second antigen that is not recognized by the TCR may not bind to the polypeptide secreted by the activated TCR-expressing cell. The second APC having a second antigen that is not recognized by the TCR may bind to a background (e.g., baseline) level of polypeptides secreted from inactivated TCR-expressing cell or may bind to a small amount of polypeptides diffused from the first APC to the second APC. In such cases, an amount of the polypeptide on the second APC may be at least about 2, 3, 4, 5, 6, 7, 8, 9, 10, 11, 12, 13, 14, 15, 16, 17, 18, 19, 20 or more times less than an amount of the polypeptide on the first APC. The second APC may be located a certain distance away from the first APC. For example, the second APC may be at least about 0.1, 0.2, 0.3, 0.4, 0.5, 0.6, 0.7, 0.8, 0.9, 1, 1.2, 1.5, 1.8, 2, 2.5, 3, 3.5, 4, 4.5, 5, 5.5, 6, 6.5, 7, 7.5, 8, 8.5, 9, 9.5, 10 or more millimeter (mm) from the first APC.

In some aspects, the present disclosure provides a method of identifying an antigen-presenting cell (APC) comprising an antigen recognized by a T-cell receptor (TCR). The method can comprise providing a plurality of antigen-presenting cells (APCs) comprising a first APC and a second APC and a plurality of TCR-expressing cells comprising the TCR. The first APC can comprise a first antigen that can be recognized by the TCR and the second APC can comprise a second antigen that may not be recognized by the TCR. Next, the first APC and the second APC can be contacted with the plurality of TCR-expressing cells. A TCR-expressing cell of the plurality can bind to the first antigen of the first APC and secrete a polypeptide upon binding. The polypeptide can bind to a first catch agent associated with the first APC. The polypeptide may not bind to a second catch agent associated with the second APC. An amount of the polypeptide on the second APC may be at least about 2, 3, 4, 5, 6, 7, 8, 9, 10, 11, 12, 13, 14, 15, 16, 17, 18, 19, 20 or more times less than an amount of the polypeptide on the first APC. Next, the polypeptide bound to the first catch agent associated with the first APC can be detected, thereby identifying the first APC as the APC comprising the antigen recognized by the TCR. The polypeptide can be diffusion-restricted. The second APC can be at least about 0.1, 0.2, 0.3, 0.4, 0.5, 0.6, 0.7, 0.8, 0.9, 1, 1.2, 1.5, 1.8, 2, 2.5, 3, 3.5, 4, 4.5, 5, 5.5, 6, 6.5, 7, 7.5, 8, 8.5, 9, 9.5, 10 or more millimeter (mm) from the first APC.

In some aspects, the present disclosure provides a method of identifying an antigen-presenting cell (APC) comprising an antigen recognized by a T-cell receptor (TCR). The method can comprise providing a plurality of antigen-presenting cells (APCs) comprising a first APC and a second APC and a plurality of TCR-expressing cells comprising the TCR. The first APC can comprise a first antigen that can be recognized by the TCR and the second APC can comprise a second antigen that may not be recognized by the TCR. Next, the first APC and the second APC can be contacted with the plurality of TCR-expressing cells. A TCR-expressing cell of the plurality can bind to the first antigen of the first APC and secrete a polypeptide (e.g., a cytokine) upon binding. The polypeptide can bind to a first catch agent associated with the first APC, and the second APC may be at least about 0.1, 0.2, 0.3, 0.4, 0.5, 0.6, 0.7, 0.8, 0.9, 1, 1.2, 1.5, 1.8, 2, 2.5, 3, 3.5, 4, 4.5, 5, 5.5, 6, 6.5, 7, 7.5, 8, 8.5, 9, 9.5, 10 or more millimeter (mm) from the first APC. The polypeptide bound to the first catch agent associated with the first APC can be detected, thereby identifying the first APC as the APC comprising the antigen recognized by the TCR. In some cases, an amount of the polypeptide on the second APC can be at least about 2, 3, 4, 5, 6, 7, 8, 9, 10, 11, 12, 13, 14, 15, 16, 17, 18, 19, 20 or more times less than an amount of the polypeptide on the first APC. The polypeptide can be diffusion-restricted.

In various methods, the first APC and the second APC of the plurality of APCs can be in the same compartment.

In some other aspects, the present disclosure provides a method for labeling an antigen-presenting cell (APC) comprising an antigen as being recognized by a T-cell receptor (TCR). The method can comprise contacting an APC comprising an antigen complexed with a major histocompatibility complex (MHC) molecule recognized by a TCR with a TCR-expressing cell comprising the TCR. The TCR-expressing cell can secrete a polypeptide upon binding the APC. The polypeptide can bind to a catch agent associated with the APC, thereby labeling the APC with the polypeptide. The catch agent can be (i) a soluble catch agent having specificity to both a cell surface protein of the APC and the polypeptide, or (ii) an exogenously expressed protein having specificity to the polypeptide. The exogenously expressed protein can be a membrane-bound protein (e.g., a transmembrane protein).

The antigen identified herein can be used to identify an additional TCR that recognizes the antigen, comprising contacting a plurality of cells, each expressing a different TCR, with the antigen. The additional TCR can be detected or selected by the cis cytokine capture assay. For example, the cells that recognize the MHC-bound antigen may be selected from those that do not. The selection may be based on binding to soluble, fluorescently labeled pMHC, pMHC tetramer or pMHC oligomer. The selection may be based on cell surface marker expression on the cells after the cells contact MHC-bound antigen. The cell surface marker may be CD25, CD69, CD39, CD10³, CD137, as well as other T cell activation markers, or the combination thereof. The selection may be based on calcium influx. The selection may also be based on reporter gene expression. The reporter gene may be a fluorescent protein (such as GFP and mCherry). The reporter gene may be under the control of a transcription factor which is regulated by TCR signaling. Examples of these transcription factors include, but are not limited to, AP-1, NFAT, NF-kappa-B, Runx1, Runx3, etc.

### Compartments

In some aspects, the methods of identifying antigens or APCs comprising the antigens can be performed in a plurality of compartments, each compartment of the plurality of compartments comprising an APC and a TCR-expressing cell. In some cases, a compartment of the plurality of compartments comprises a single APC and one or more TCR-expressing cells. In some cases, a compartment of the plurality of compartments comprises a single APC and a single TCR-expressing cell. The plurality of compartments can comprise at least about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more compartments.

Methods of partitioning biological particles (e.g., cells) include, for example, microfluidics based methods and non-microfluidics based methods (e.g., vortexing). The present disclosure provides methods comprising partitioning biological particles into compartments so that in some compartments there is only one biological particle (e.g., an APC) in a compartment.

In some embodiments, at least about 50%, 60%, 70%, 75%, 80%, 85%, 90%, 95%, 96%, 97%, 98%, 99%, or 100% of the compartments contain zero or only one biological particle. In some embodiments, at least about 50%, 60%, 70%, 75%, 80%, 85%, 90%, 95%, 96%, 97%, 98%, 99%, or 100% of the compartments contain zero or only one primer delivery particle.

The number of partitions or compartments employed can vary depending on the application. For example, the number of partitions or compartments can be about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be less than 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be about 5-10000000, 5-5000000, 5-1000000, 10-10000, 10-5000, 10-1000, 1000-6000, 1000-5000, 1000-4000, 1000-3000, or 1000-2000.

The number of biological particles (e.g., APCs or TCR-expressing cells) that are partitioned into compartments can be about 1, 2, 3, 4, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of biological particles that are partitioned into compartments can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of biological particles that are partitioned into compartments can be less than 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of biological particles that are partitioned into compartments can be about 5-10000000, 5-5000000, 5-1000000, 10-10000, 10-5000, 10-1000, 1000-6000, 1000-5000, 1000-4000, 1000-3000, or 1000-2000.

In some embodiments, the compartments can be wells in a standard microwell plate with separation aided by sorting. In some embodiments, the sorter can be a fluorescence activated cell sorter (FACS). Additionally, partitioning can be coupled with automated library generation in separated microfluidics chambers, as is the case with the Fluidigm C1. In some embodiments, the partition is a subnanoliter well and biological particles can be sealed by a semipermeable membrane.

### Compositions

The present disclosure also provides a compartment comprising a TCR-expressing cell comprising a T-cell receptor (TCR); and an antigen-presenting cell (APC) associated with a catch agent. The catch agent may be capable of binding to a polypeptide such as a cytokine secreted by the TCR-expressing cell. The catch agent can be (i) a soluble catch agent having specificity to both a cell surface protein of the APC and the polypeptide that is secreted, or (ii) an exogenously expressed protein having specificity to the polypeptide. The exogenously expressed protein can be a membrane-bound protein. The membrane-bound protein can be a receptor. The membrane-bound protein can be a cytokine receptor (e.g., an IL-2 receptor).

The soluble catch agent can be (i) a molecule expressed and/or secreted by the APC or the TCR-expressing cell or another cell supplied (e.g., co-partitioned) within the compartment or (ii) a molecule supplied (e.g., co-partitioned) within the compartment. The soluble catch agent can have specificity to a cell surface protein of the APC and the polypeptide. The soluble catch agent can bind to both a cell surface protein of the APC and the polypeptide.

The cell surface protein can be an endogenous protein or an exogenous protein of the APC. The soluble catch agent can be an antibody or a fragment thereof. The antibody can be a bispecific antibody (BsAb). The compartment can comprise a plurality of compartments, each compartment of the plurality of compartments comprising a TCR-expressing cell and an APC.

The compartment can be a well, a microwell, a hole, a tube, a capsule, or a droplet. In some cases, the compartment is a droplet (e.g., water-in-oil droplet). In some cases, the compartment is a well. The compartment may comprise a plurality of TCR-expressing cells or a plurality of APCs. The compartment may further comprise a plurality of polymerizable or gellable polymers or monomers. The plurality of polymerizable or gellable polymers or monomers can be polymerized or gelled such that the cells or molecules (e.g., secreted cytokines) can be diffusion-restricted.

### Pharmaceutical Compositions

The present disclosure also provides pharmaceutical compositions comprising one or more antigens identified using the methods described herein in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Examples of carriers or excipients can include dextrose, sodium chloride, sucrose, lactose, cellulose, xylitol, sorbitol, malitol, gelatin, PEG, PVP, and any combination thereof. Compositions of the present disclosure can be formulated for intravenous administration. In some instances, the pharmaceutical composition described herein can be administered by a route selected from subcutaneous injection, intramuscular injection, intradermal injection, percutaneous administration, intravenous (“i.v.”) administration, intranasal administration, intralymphatic injection, and oral administration.

Pharmaceutical compositions of the present disclosure may be administered in a manner appropriate to the disease to be treated (or prevented). The quantity and frequency of administration can be be determined by such factors as the condition of the patient, and the type and severity of the patient's disease, although appropriate dosages may be determined by clinical trials.

The pharmaceutical composition can be substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some cases, the bacterium can be at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, Streptococcus pyogenes group A, and any combinations thereof.

### EXAMPLES

### Example 1: Detecting Jurkat Activation in Trans in Droplets

This example shows a trans cytokine catch assay with Jurkat T cell line (JK1) as a model for cytokine secreting cell and K562 as a model for APCs. Instead of using antigen presentation as the activation stimulus for the JK1, Dynabeads and PMA were used to chemically induce activation. The cytokine catch assay itself was performed with the Human IL-2 Secretion Assay, allophycocyanin kit (Miltenyi, #130-090-763). K562 were labeled with the kit catch reagent to perform the cytokine catch in trans.

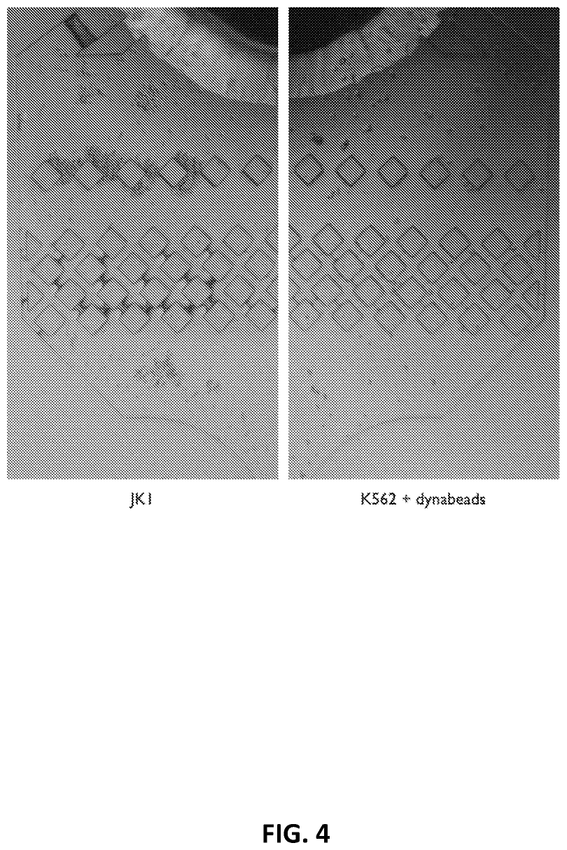

A two-inlet microfluidics chip was used to generate droplets in this example (FIG. 4). One inlet contained PMA and the JK1 cells. The other contained the labeled K562 and the Dynabeads. The overall proportion of entities in this experiment was five JK1 cells to one K562 cells to three Dynabeads. These two mixes were flowed through the microfluidics chip to co-encapsulate the JK1, K562, and Dynabeads in a single droplet, aiming for single occupancy of the K562. Since the JK1 and Dynabeads were in excess of the K562, they were present in multiples along with the single K562 cell. In addition to generating droplets with these mixtures, a simple bulk sample from combining the two mixes was also set up on the side for control comparison.

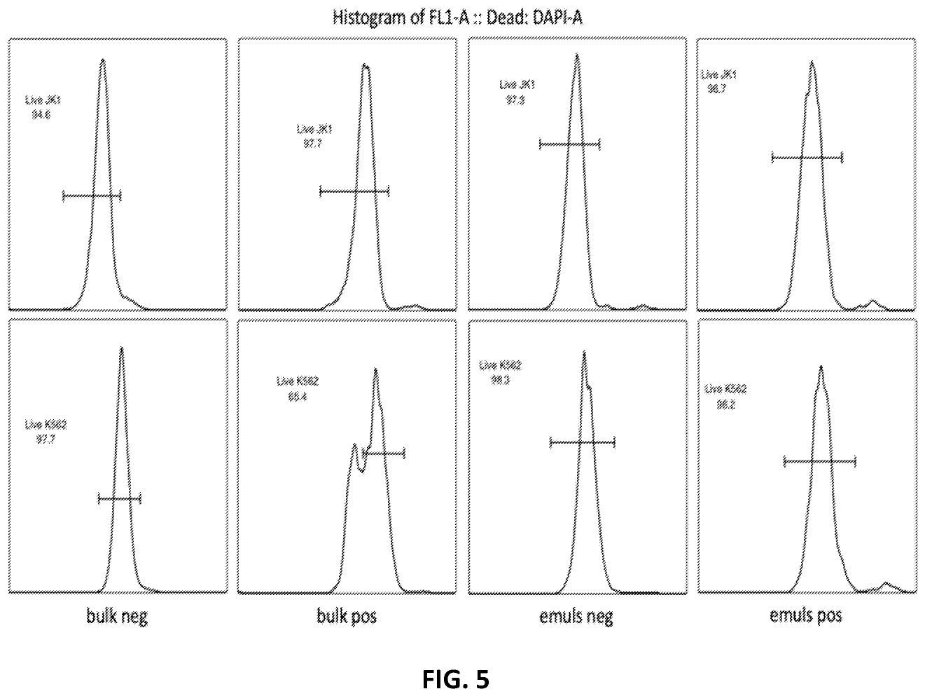

After encapsulation, both the bulk and emulsion mixtures were incubated at 37° C. overnight to allow for sufficient time for JK1 activation, cytokine secretion, and cytokine catch. After incubation, the emulsion samples were demulsified, and all samples were stained with DAPI, CD3 antibody, and the Miltenyi detection reagent before analysis by flow cytometry. DAPI staining was used to investigate the viability of cells after the overnight incubation and handling in emulsion (FIG. 5). Comparing both negative (no activation; as indicated by “neg”) against positive (activation; as indicated by “pos”) as well as bulk against emulsion showed no significant shift in cell viability over the course of the assay. The “bulk neg” indicates the mixture in bulk with no activation. The “bulk pos” indicates the mixture in bulk with activation. The “emuls neg” indicates the mixture in emulsion with no activation. The “emuls pos” indicates the mixture in emulsion with activation.

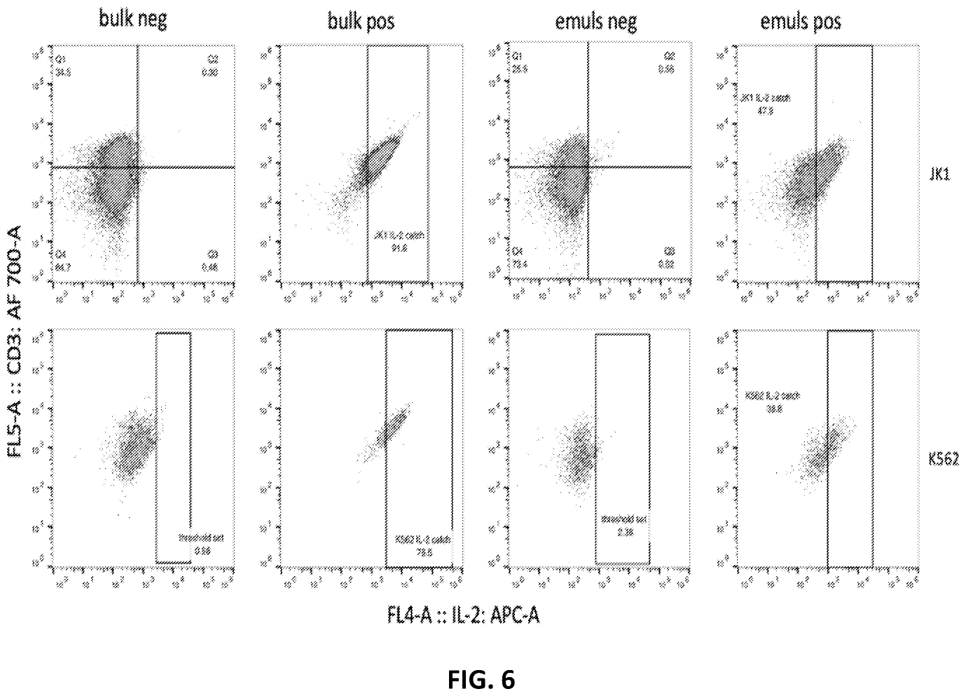

The Miltenyi detection reagent used allophycocyanin as its marker (FIG. 6). Detection reagent were expected to only be present where there was successfully caught IL-2 with the catch reagent, which in turn serves as an indicator for the secretion of IL-2 by an activated JK1 cell in the same compartment. The negative condition without activation in the control bulk populations provided the baseline allophycocyanin fluorescence of just the cells without any secretion. Comparison with the positive condition that included the activation agents revealed a significant shift in allophycocyanin fluorescence. This shift represented the representative readout of IL-2 secretion from JK1 through the chosen activation method detected in the base condition. For the test emulsion populations, the shift in allophycocyanin fluorescence between the negative and positive activation conditions was mirrored, although in a smaller proportion of the cell population. This showed that IL-2 secretion from JK1 in a droplet can be detected, albeit at lower levels.

### Example 2: Labeling of APCs with Cytokine Capturing Antibody for Identification of Specific T Cell Antigens

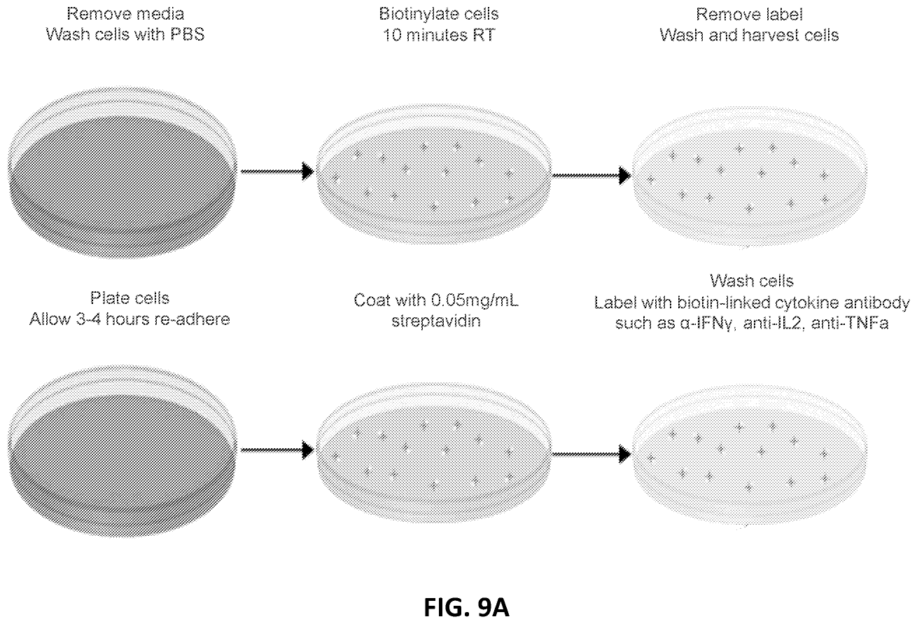

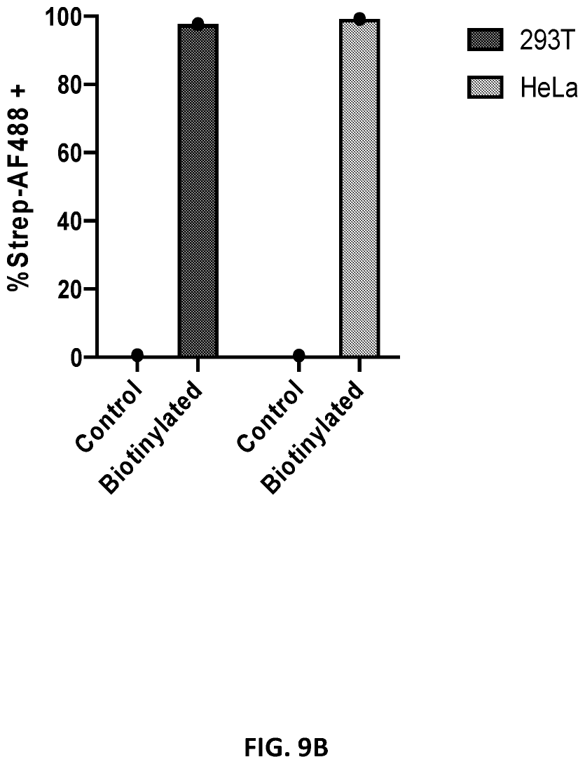

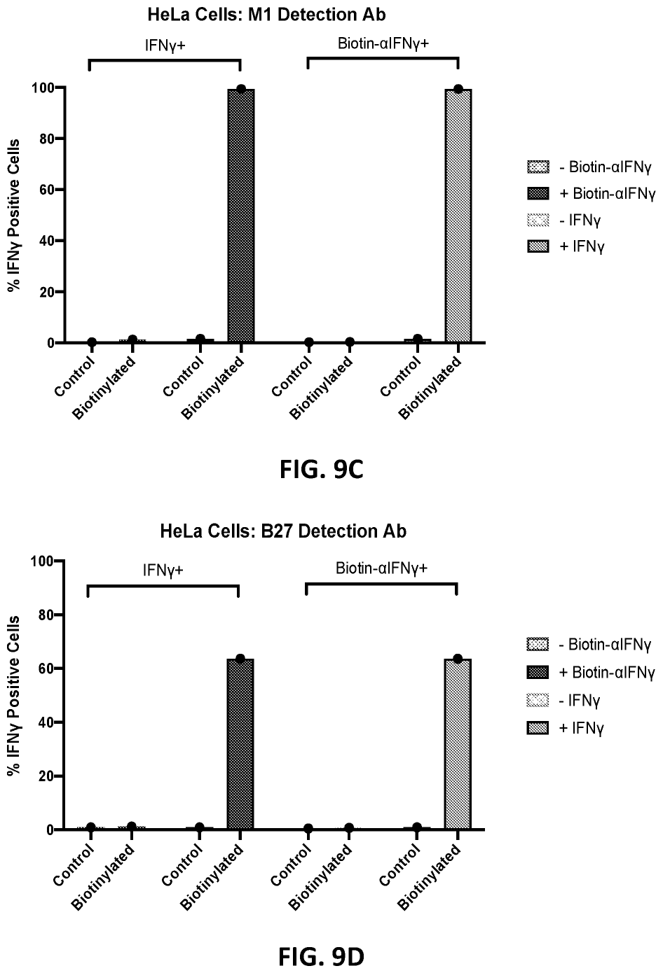

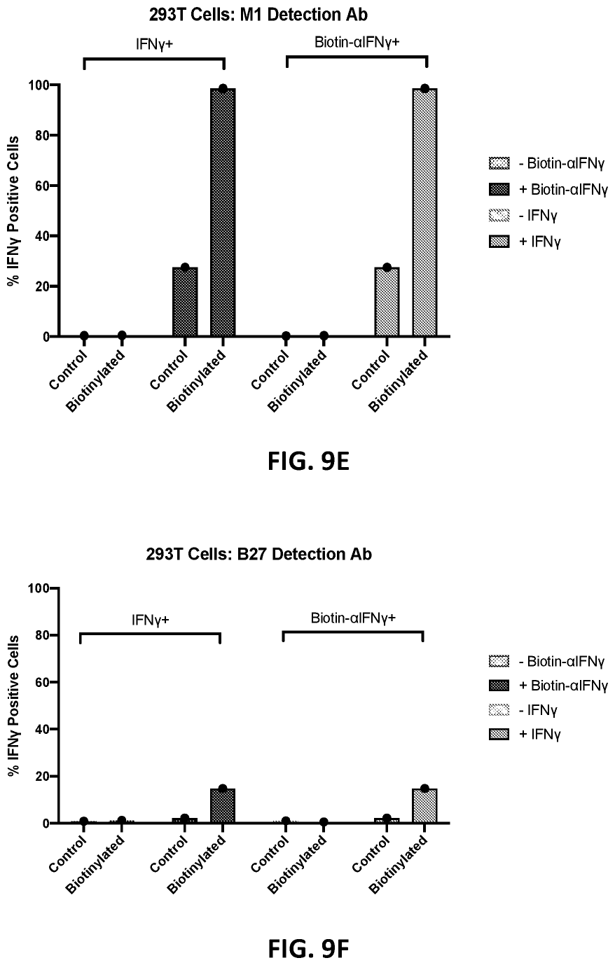

T cells, via their T cell receptor (TCR), can bind antigen presented in the context of MHC in a highly specific manner. A T cell can become activated and release cytokines when the TCR interacts with the correct HLA-antigen complex on an antigen presenting cell (APC). The example described herein shows the method to label antigen presenting cells (APCs) with a cytokine capturing antibody such as interferon gamma (IFNγ), IL-2 or TNFα, that upon correct antigen recognition, the T cell can be activated and release cytokines that will then be captured on the APC by the previously attached capture antibody. Once the cytokine is captured by the APC it can then be detected by a fluorescent cytokine detection antibody. This will allow sorting the cytokine positive APCs by fluorescent activation cell sorting (FACS). The APCs can be engineered to express one antigen exogenously that can be used to specifically sequence the epitope recognized by the T cell. Although cytokines can defuse in the culture media and label other APCs non-specifically, this labeling can be a function of distance from the true antigen positive APC. Sequencing multiple samples of the same T cell-APC co-culture allows the non-specific noise to dropout as that will be random and not captured in replicate cultures if the diversity of the antigen library is large. FIG. 9A shows a summary of the method for labeling APCs with a cytokine capturing antibody to detect antigen-specific T cells along with their cognate antigen.

In this example, both 293T and HeLa cells were biotinylated in culture (FIG. 9B), and then coated with unlabeled streptavidin. FIG. 9B shows that biotinylated 293T and HeLa cells were stained positive for Strep-AF488 versus control cells. Next, the cells were treated with a biotin conjugated antibody specific for IFNγ (clone 4S.B3, “Biotin-αIFNγ”). The cells were then treated with recombinant human IFNγ overnight, and the cells were stained with detection antibodies for IFNγ the following day (clones B27 and M1).

FIG. 9C shows that HeLa cells specifically captured IFNγ in the presence of both Biotin-αIFNγ and IFNγ. The detection antibody used here was clone M1. FIG. 9E shows that 293T cells specifically captured IFNγ in the presence of both Biotin-αIFNγ and IFNγ. 293T cells had some background capture of interferon on control (non-biotinylated) cells. Similar experiments were conducted for both HeLa cells and 293T cells using clone B27 as the detection antibody (FIG. 9D and FIG. 9F). The B27 clone for anti-IFNγ appeared to be less sensitive for detection of interferon, and perhaps competed with the 4S.B3 clone used as the capturing antibody.

### Experiment 3: Two Color Labeling of WT Vs. HLA-A02:01 Presenting APCs to Demonstrate Specific Labeling of APCs in Co-Culture with a Model TCR T Cell

As described herein, identification of a TCRs' specific antigen can be beneficial for the development of T cell-based therapies. By enabling antigen identification, safety of a TCR could be addressed in addition to discovery of potential “off the shelf” TCRs (e.g., a TCR that can be used for multiple patients or indications). In this example, a two-color labeling co-culture assay was used with a cytokine catch reagent in place of the biotin-labeled APC. This assay may not lead to non-specific labeling of surrounding APCs which are not recognized by the T cells. The catch reagent can comprise two monoclonal antibodies linked at their Fc regions. One antibody can bind specifically to CD45 at the surface of immune cells, while the other antibody can be the “catch” antibody which specifically binds cytokines secreted by T cells that are in close contact with the APC. In this assay, K562 cells, a leukemia derived immortalized tumor cell line, were used as the APC in co-cultures with T cells. K562 cells can be a useful tool in these assays because the wildtype (WT) cell line is negative for surface expression of MHC-I, and can be electroporated with specific MHC-I genes such as HLA-A02:01. Alternatively, a cell line with stabilized surface expression of a mixture of HLA-A02:01 clones (K562-A2) was developed which can be pulsed with model peptides, or electroporated with tandem minigenes (TMG) that can be presented to T cells for recognition via their TCR.

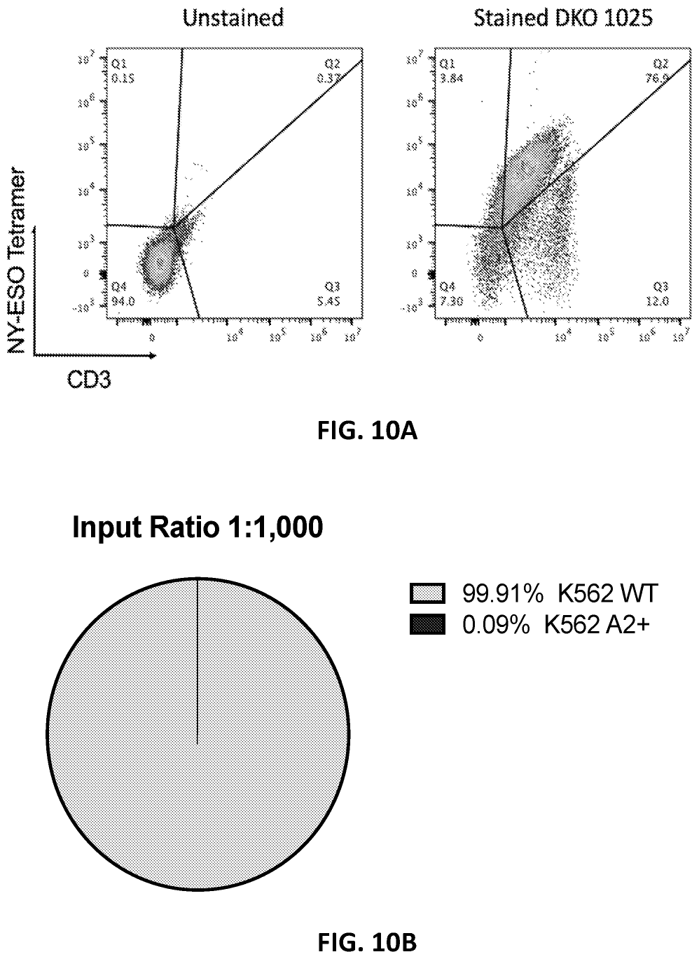

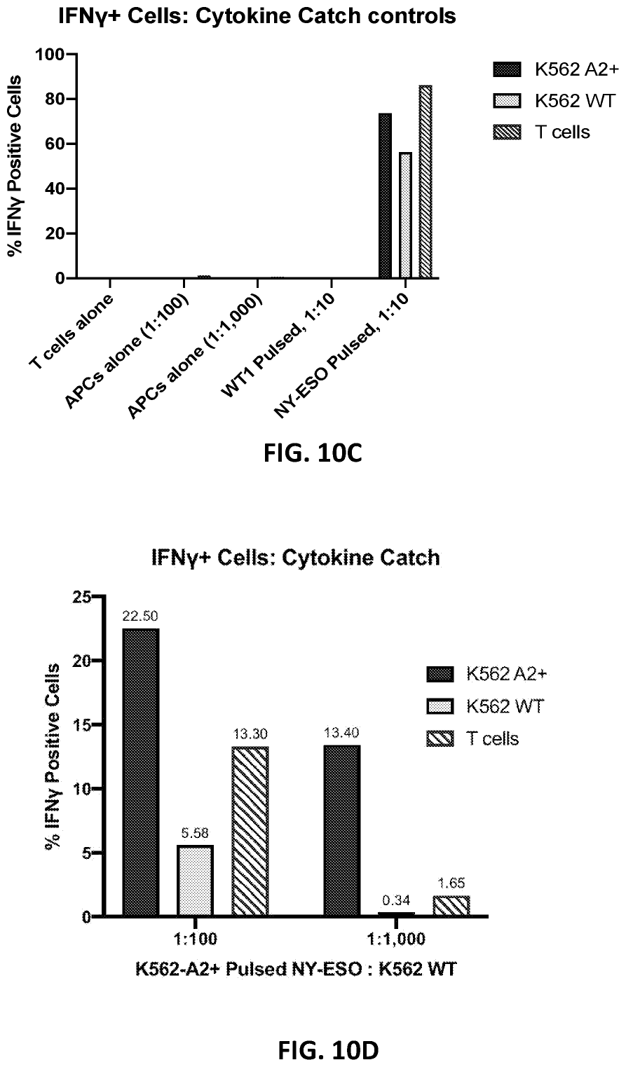

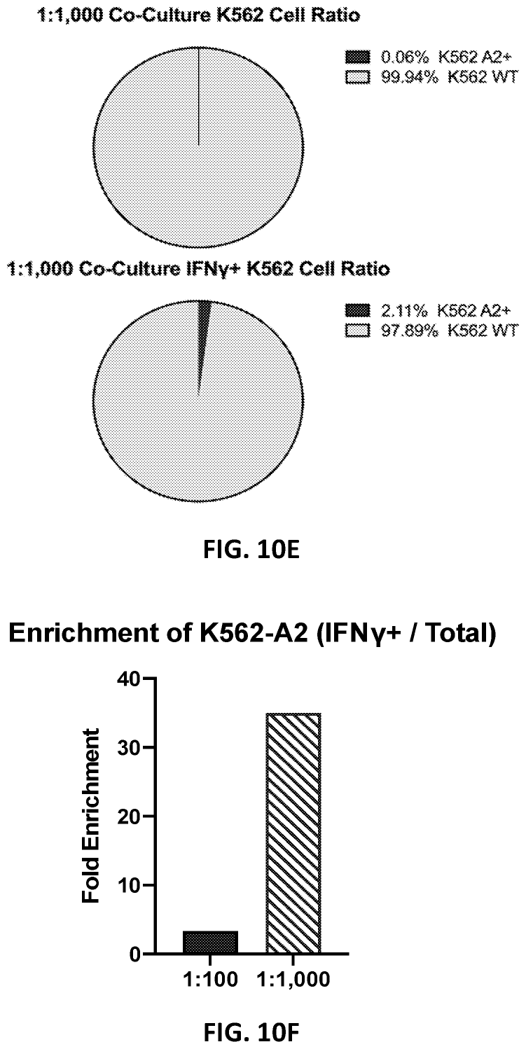

In this experiment, double knockout (DKO) T cells were generated from human donors by electroporating cells with CRISPR/Cas9 targeting the TCRα and TCRβ genes that together form the T cell receptor at the surface. These DKO T cells are also negative for CD3 at the surface, since TCRαβ pairing can be required for CD3 shuttling and surface expression. The DKO T cells were next electroporated with a model TCR specific for a model antigen, in this case, the TCR for NY-ESO. Cells were then stained with tetramer and CD3 to confirm expression of the model TCR at the surface (FIG. 10A). Next, the APCs were prepared for co-culture. K562-WT cells in excess (1:1,000 ratio) of K562-A2 cells were pulsed with the model peptide (NY-ESO) versus control peptide (WT1). The APCs were cell trace labeled with two different colors to distinguish them via flow cytometry (input ratio of APCs shown in FIG. 10B).

The color-labeled cells were then labeled with an IFNγ cytokine catch reagent which binds to CD45 at the surface of immune cells. The labeled APCs were then co-cultured with model TCR T cells overnight. T cells, upon recognition of their cognate antigen on the APC, can produce IFNγ locally, which can be captured by the IFNγ catch reagent. Only APCs that present model peptide of NY-ESO to T cells can be labeled with IFNγ. The following day, cells were collected and bulk-stained for IFNγ to determine the fold enrichment of labeling. Control culture conditions included culturing the T cells alone, the APCs alone (both 1:100 and 1:1,000 ratio), and K562-A2 cells pulsed with control peptide of WT1. All negative control conditions failed to produce cells positive for IFNγ (FIG. 10C). At high K562-A2:K562-WT ratios of 1:10, there was some background labeling of K562-WT cells (FIG. 10C). FIG. 10D shows percent IFNγ positive cells in cell culture conditions where the ratio of K562-A2 pulsed with NY-ESO:K562-WT is 1:100 or 1:1000. The results show that K562-A2+ pulsed with NY-ESO, which can be recognized by the T cells expressing TCRs for NY-ESO, had relatively high percentage of IFNγ positive cells, while K562-WT cells had only a background level of labeling. Following co-culture, APC cells that are positive for IFNγ were highly enriched for K562-A2, suggesting specific labeling of APCs for the model TCR peptide (FIG. 10D and FIG. 10E). The total fold enrichment of K562-A2 cells in the 1:1,000 co-culture condition observed was greater than 30-fold (FIG. 10F). The experiments described herein in Example 2 and 3 were performed with APCs in suspension or adherent. The cell density of cells in suspension were placed at about 2.5×10⁵ cells/mL. The cell density of adherent cells were placed at about 10⁴ to 10⁵ cells/mL. Similar experiments can be performed within individual compartments such as water-in-oil droplets.

While preferred embodiments of the present disclosure have been shown and described herein, it will be obvious to those skilled in the art that such embodiments are provided by way of example only. It is not intended that the inventions of the present disclosure be limited by the specific examples provided within the specification. While embodiments of the present disclosure have been described with reference to the aforementioned specification, the descriptions and illustrations of the embodiments herein are not meant to be construed in a limiting sense. Numerous variations, changes, and substitutions will now occur to those skilled in the art without departing from the scope of the present disclosure. Furthermore, it shall be understood that all aspects of the inventions of the present disclosure are not limited to the specific depictions, configurations or relative proportions set forth herein which depend upon a variety of conditions and variables. It should be understood that various alternatives to the embodiments of the present disclosure described herein may be employed in practicing the inventions of the present disclosure. It is therefore contemplated that the present disclosure shall also cover any such alternatives, modifications, variations or equivalents. It is intended that the following claims define the scope of the inventions of the present disclosure and that methods and structures within the scope of these claims and their equivalents be covered thereby.

What is claimed is:

1. A method of identifying an antigen-presenting cell (APC) comprising an antigen recognized by a T-cell receptor (TCR), the method comprising:
(a) providing a plurality of APCs, each comprising a different antigen complexed with a major histocompatibility complex (MHC) molecule, and a plurality of TCR-expressing cells comprising a TCR;
(b) partitioning the plurality of APCs and the plurality of TCR-expressing cells into a plurality of compartments, a compartment of the plurality of compartments comprising (i) an APC of the plurality of APCs and (ii) at least one TCR-expressing cell of the plurality of TCR-expressing cells;
(c) binding an antigen complexed with an MHC molecule of the APC to the TCR of the at least one TCR-expressing cell within the compartment, wherein the at least one TCR-expressing cell secretes a polypeptide upon binding, and wherein the polypeptide that is secreted binds to a catch agent associated with the APC; and
(d) selecting the APC based on the polypeptide bound to the catch agent associated with the APC, thereby identifying the APC comprising the antigen recognized by the TCR.

2. A method of identifying an antigen-presenting cell (APC) comprising an antigen recognized by a T-cell receptor (TCR), the method comprising:
(a) providing a plurality of APCs, each comprising a different antigen complexed with a major histocompatibility complex (MHC) molecule, and a plurality of TCR-expressing cells comprising a TCR;
(b) partitioning the plurality of APCs and the plurality of TCR-expressing cells into a plurality of compartments, a compartment of the plurality of compartments comprising (i) an APC of the plurality of APCs and (ii) at least one TCR-expressing cell of the plurality of TCR-expressing cells;
(c) binding an antigen complexed with an MHC molecule of the APC to the TCR of the at least one TCR-expressing cell within the compartment, wherein the at least one TCR-expressing cell secretes a polypeptide upon binding, and wherein the polypeptide that is secreted binds to a catch agent associated with the APC; and
(d) detecting the polypeptide bound to the catch agent associated with the APC, thereby identifying the APC comprising the antigen recognized by the TCR.

3. The method of claim 2, wherein detecting in (d) comprises contacting the polypeptide bound to the catch agent associated with the APC with a detection agent, wherein the detection agent binds to the polypeptide.

4. The method of any one of claims 1-3, wherein the polypeptide is endogenous or exogenous of the at least one TCR-expressing cell.

5. The method of any one of claims 1-4, wherein the polypeptide is engineered to be secreted by the at least one TCR-expressing cell.

6. The method of any one of claims 1-5, wherein the polypeptide is a cytokine.

7. The method of any one of claims 1-6, wherein the catch agent is associated with the APC indirectly.

8. The method of claim 7, wherein the compartment of the plurality of compartments of (b) further comprises a solid support.

9. The method of claim 8, wherein the solid support is associated with the APC.

10. The method of claim 9, wherein the solid support is modified with an antibody or fragment thereof, which antibody or fragment thereof binds to a cell surface protein of the APC.

11. The method of any one of claims 8-10, wherein the catch agent is associated with the solid support.

12. The method of claim 11, wherein the catch agent is associated with the solid support via a crosslinker or an affinity binding pair.

13. The method of any one of claims 8-12, wherein the solid support is a bead or a hydrogel particle.

14. The method of any one of claims 1-6, wherein the catch agent is associated with the APC directly.

15. The method of claim 14, wherein the catch agent is associated with the APC directly via covalent or non-covalent interaction.

16. The method of claim 15, wherein the catch agent is associated with the APC via click chemistry.

17. The method of claim 16, wherein the catch agent comprises a conjugation handle, and the APC comprises an additional conjugation handle that reacts with the conjugation handle via click chemistry.

18. The method of claim 17, wherein the conjugation handle and the additional conjugation handle comprise TCO and tetrazine, azide and alkyne, or azide and DBCO.

19. The method of claim 15, wherein the catch agent is associated with the APC via an affinity binding pair.

20. The method of claim 19, wherein the affinity binding pair comprises streptavidin and biotin.

21. The method of any one of claims 1-17, wherein the catch agent is a membrane-bound catch agent.

22. The method of claim 21, wherein the membrane-bound catch agent is a transmembrane protein.

23. The method of claim 22, wherein the transmembrane protein is a receptor expressed by the APC.

24. The method of claim 23, wherein the receptor is exogenously expressed by the APC.

25. The method of any one of claims 1-17, wherein the catch agent is a soluble catch agent.

26. The method of claim 25, wherein the soluble catch agent is (i) a molecule expressed and/or secreted by the APC or the TCR-expressing cell or (ii) a molecule supplied within the compartment.

27. The method of claim 25 or 26, wherein the soluble catch agent has specificity to a cell surface protein of the APC and the polypeptide.

28. The method of any one of claims 25-27, wherein the soluble catch agent binds to both a cell surface protein of the APC and the polypeptide.

29. The method of claim 28, wherein the cell surface protein is an endogenous protein or an exogenous protein of the APC.

30. The method of any one of claims 25-29, wherein the soluble catch agent is an antibody or a fragment thereof.

31. The method of claim 30, wherein the antibody is a bispecific antibody (BsAb).

32. The method of claim 31, wherein the BsAb is an antibody produced by a quadroma cell.

33. The method of claim 31 or 32, wherein the BsAb is a heterodimeric antibody or a fragment thereof.

34. The method of claim 31, wherein the BsAb is a recombinant protein or a bispecific fusion protein.

35. The method of claim 31, wherein the BsAb comprises a first antigen binding domain targeting the cell surface protein of the APC and a second antigen binding domain targeting the polypeptide.

36. The method of claim 35, wherein the first antigen binding domain and the second antigen binding domain are linked by a linker.

37. The method of claim 36, wherein the linker is a chemical linker.

38. The method of any one of claims 35-37, wherein the first antigen binding domain or the second antigen binding domain is an IgG, a scFv or a sdAb.

39. The method of any one of claims 27-38, wherein the cell surface protein is CD11b, CD11c, CD14, CD80, CD86, B7-1, B7-2, CD18, CD29, CD31, CD40, CD43, CD44, CD45, CD54, CD58, CD83, CD86, IFN-γ receptor, IL-2 receptor, ICAM-1, Fcγ receptor, CMRF-44, CMRF-56, DCIR, or DECTIN-1.

40. The method of any one of claims 1-39, wherein the polypeptide is TNFα tumor necrosis factor alpha (TNFα), TNFβ, interleukin (IL)-1α, IL-1β, IL-2, IL-4, IL-5, IL-6, IL-9, IL-10, IL-12, IL-13, IL-15, IL-17, IL-18, IL-21, IL-22, IL-25, transforming growth factor beta (TGF-β), or interferon (IFN-γ).

41. The method of claim 40, wherein the cell surface protein is CD45, and wherein the polypeptide is selected from the group consisting of IL-2, IL-4, IL-10, IL-12, IFN-γ and TNFβ.

42. The method of claim 41, wherein the polypeptide is IL-2.

43. The method of claim 40, wherein the cell surface protein is CD11b, and wherein the polypeptide is selected from the group consisting of IL-2, IL-4, IL-10, IL-12, IFN-γ, TNFα and TNFβ.

44. The method of claim 43, wherein the polypeptide is IFN-γ.

45. The method of any one of claims 1 and 4-44, wherein selecting in (d) comprises contacting the polypeptide bound to the catch agent associated with the APC with a detection agent, wherein the detection agent binds to the polypeptide.

46. The method of claim 3 or 45, wherein the detection agent is an antibody or fragment thereof.

47. The method of any one of claims 3, 45 and 46, wherein the detection agent has specificity to the polypeptide.

48. The method of any one of claims 3 and 45-47, wherein the detection agent and the catch agent bind to different epitopes of the polypeptide.

49. The method of any one of claims 3 and 45-47, wherein the detection agent comprises a signal or an affinity tag.

50. The method of claim 49, wherein the signal is a detectable label.

51. The method of claim 50, wherein the detectable label is a fluorescent label.

52. The method of any one of claims 1-51, further comprising, subsequent to (c), releasing the plurality of APCs from the plurality of compartments, wherein the APC bound with the polypeptide is released from the compartment.

53. The method of claim 52, wherein the plurality of compartments is a plurality of droplets, and wherein releasing comprises demulsifying the plurality of droplets.

54. The method of any one of claims 49-53, wherein selecting comprises selecting the APC comprising the antigen recognized by the TCR based on the signal or the affinity tag of the detection agent.

55. The method of claim 54, wherein selecting comprises sorting the plurality of APCs by flow cytometry based on the signal from the detection agent, and wherein the APC comprising the antigen recognized by the TCR is selected.

56. The method of claim 54, wherein selecting comprises capturing the affinity tag of the detection agent.

57. The method of any one of claims 1-56, wherein the APC comprises a nucleic acid molecule encoding the antigen.

58. The method of claim 57, further comprising sequencing the nucleic acid or a derivative thereof to identify the antigen.

59. The method of any one of claims 1-58, wherein the plurality of APCs comprises artificial APCs (aAPCs).

60. The method of claim 59, wherein the aAPCs comprises cells engineered to express an MHC molecule.

61. The method of claim 60, wherein the cells engineered to express an MHC molecule is a cell line or cells isolated from a subject.

62. The method of claim 61, wherein the cell line is K562.

63. The method of claim 61, wherein the cells isolated from a subject are tumor cells.

64. The method of any one of claims 1-58, wherein the plurality of APCs comprises professional APCs or non-professional APCs.

65. The method of claim 64, wherein the professional APCs comprise dendritic cells, macrophages, or B cells.

66. The method of claim 64, wherein the non-professional APCs comprise fibroblasts, thymic epithelial cells, thyroid epithelial cells, glial cells, pancreatic beta cells, or vascular endothelial cells.

67. The method of any one of claims 1-66, wherein the plurality of TCR-expressing cells comprises T cells.

68. The method of claim 67, wherein the T cells comprise a CD4+ or CD8+ T cell.

69. The method of claim 67, wherein the T cells comprise a cytotoxic T cell, an ancillary T cell, a natural killer T cell, an alpha beta T cell, a gamma delta T cell, a regulatory T cell or a memory T cell.

70. The method of claim 67, wherein the T cells are a cell line.

71. The method of any one of claims 1-70, wherein the compartment of the plurality of compartments comprises a single APC.

72. The method of any one of claims 1-71, wherein each compartment of the plurality of compartments comprises a single APC.

73. The method of any one of claims 1-72, wherein the plurality of compartments comprises at least about 20, 100, 1,000, 10,000, 100,000 or more compartments.

74. The method of any one of claims 1-73, wherein the plurality of TCRs comprises at least about 2 times more cells than the plurality of APCs.

75. A method of identifying an antigen-presenting cell (APC) comprising an antigen recognized by a T-cell receptor (TCR), comprising:
(a) providing (i) a plurality of antigen-presenting cells (APCs) comprising a first APC and a second APC, wherein the first APC comprises a first antigen that is recognized by the TCR and the second APC comprises a second antigen that is not recognized by the TCR, and (ii) a plurality of TCR-expressing cells comprising the TCR;
(b) contacting the first APC and the second APC with the plurality of TCR-expressing cells, wherein a TCR-expressing cell of the plurality binds to the first antigen of the first APC and secretes a polypeptide upon binding, wherein the polypeptide that is secreted binds to a first catch agent associated with the first APC, and wherein the polypeptide is diffusion-restricted such that it does not bind to a second catch agent associated with the second APC; and
(c) detecting the polypeptide bound to the first catch agent associated with the first APC, thereby identifying the first APC as the APC comprising the antigen recognized by the TCR.

76. The method of claim 75, wherein an amount of the polypeptide on the second APC is at least 5 times less than an amount of the polypeptide on the first APC.

77. The method of claim 75 or 76, wherein the second APC is at least about 0.1 mm from the first APC.

78. A method of identifying an antigen-presenting cell (APC) comprising an antigen recognized by a T-cell receptor (TCR), comprising:
(a) providing (i) a plurality of antigen-presenting cells (APCs) comprising a first APC and a second APC, wherein the first APC comprises a first antigen that is recognized by the TCR and the second APC comprises a second antigen that is not recognized by the TCR, and (ii) a plurality of TCR-expressing cells comprising the TCR;
(b) contacting the first APC and the second APC with the plurality of TCR-expressing cells, wherein a TCR-expressing cell of the plurality binds to the first antigen of the first APC and secretes a polypeptide upon binding, wherein the polypeptide binds to a first catch agent associated with the first APC, and wherein an amount of the polypeptide on the second APC is at least 5 times less than an amount of the polypeptide on the first APC; and
(c) detecting the polypeptide bound to the first catch agent associated with the first APC, thereby identifying the first APC as the APC comprising the antigen recognized by the TCR.

79. The method of claim 78, wherein the polypeptide is diffusion-restricted.

80. The method of claim 78 or 79, wherein the second APC is at least about 0.1 mm from the first APC.

81. A method of identifying an antigen-presenting cell (APC) comprising an antigen recognized by a T-cell receptor (TCR), comprising:
(a) providing (i) a plurality of antigen-presenting cells (APCs) comprising a first APC and a second APC, wherein the first APC comprises a first antigen that is recognized by the TCR and the second APC comprises a second antigen that is not recognized by the TCR, and (ii) a plurality of TCR-expressing cells comprising the TCR;
(b) contacting the first APC and the second APC with the plurality of TCR-expressing cells, wherein a TCR-expressing cell of the plurality binds to the first antigen of the first APC and secretes a polypeptide upon binding, wherein the polypeptide binds to a first catch agent associated with the first APC, and wherein the second APC is at least about 0.1 mm from the first APC; and
(c) detecting the polypeptide bound to the first catch agent associated with the first APC, thereby identifying the first APC as the APC comprising the antigen recognized by the TCR.

82. The method of claim 81, wherein an amount of the polypeptide on the second APC is at least 5 times less than an amount of the polypeptide on the first APC.

83. The method of claim 81 or 82, wherein the polypeptide is diffusion-restricted.

84. The method of any one of claims 75-83, wherein the polypeptide is endogenous or exogenous of the TCR-expressing cell.

85. The method of any one of claims 75-84, wherein the polypeptide is engineered to be secreted by the TCR-expressing cell.

86. The method of any one of claims 75-85, wherein the polypeptide is a cytokine.

87. The method of any one of claims 75-86, wherein the first APC and the second APC are in the same compartment.

88. The method of any one of claims 75-84, wherein the first or the second catch agent has specificity to the polypeptide.

89. The method of any one of claims 75-88, wherein the first or the second catch agent is associated with the APC indirectly.

90. The method of claim 89, wherein the compartment of the plurality of compartments of (b) further comprises a solid support.

91. The method of claim 90, wherein the solid support is associated with the APC.

92. The method of claim 91, wherein the solid support is modified with an antibody or fragment thereof, which antibody or fragment thereof binds to a cell surface protein of the APC.

93. The method of any one of claims 90-92, wherein the first or the second catch agent is associated with the solid support.

94. The method of claim 93, wherein the first or the second catch agent is associated with the solid support via a crosslinker or an affinity binding pair.

95. The method of any one of claims 90-94, wherein the solid support is a bead or a hydrogel particle.

96. The method of any one of claims 75-88, wherein the first or the second catch agent is associated with the APC directly.

97. The method of claim 96, wherein the first or the second catch agent is associated with the APC directly via covalent or non-covalent interaction.

98. The method of claim 97, wherein the first or the second catch agent is associated with the APC via click chemistry.

99. The method of claim 98, wherein the first or the second catch agent comprises a conjugation handle, and the APC comprises an additional conjugation handle that reacts with the conjugation handle via click chemistry.

100. The method of claim 99, wherein the conjugation handle and the additional conjugation handle comprise TCO and tetrazine, azide and alkyne, or azide and DBCO.

101. The method of claim 97, wherein the first or the second catch agent is associated with the APC via an affinity binding pair.

102. The method of claim 101, wherein the affinity binding pair comprises streptavidin and biotin.

103. The method of any one of claims 75-102, wherein the first or the second catch agent is a membrane-bound catch agent.

104. The method of claim 103, wherein the membrane-bound catch agent is a transmembrane protein.

105. The method of claim 104, wherein the transmembrane protein is a receptor expressed by the first or the second APC.

106. The method of claim 105, wherein the receptor is exogenously expressed.

107. The method of any one of claims 75-102, wherein the first or the second catch agent is a soluble catch agent.

108. The method of claim 107, wherein the soluble catch agent is (i) a molecule expressed and/or secreted by the first or the second APC or the TCR-expressing cell or (ii) a molecule supplied within the same compartment.

109. The method of claim 107 or 108, wherein the soluble catch agent has specificity to a cell surface protein of the first APC and the polypeptide.

110. The method of any one of claims 107-109, wherein the soluble catch agent binds to both a cell surface protein of the first APC and the polypeptide.

111. The method of claim 110, wherein the cell surface protein is an endogenous protein or an exogenous protein of the first APC.

112. The method of any one of claims 107-111, wherein the soluble catch agent is an antibody or a fragment thereof.

113. The method of claim 112, wherein the antibody is a bispecific antibody (BsAb).

114. The method of any one of claims 75-113, wherein detecting in (c) comprises contacting the polypeptide bound to the first catch agent associated with the APC with a detection agent, wherein the detection agent binds to the polypeptide.

115. The method of claim 114, wherein the detection agent is an antibody or fragment thereof.

116. The method of claim 114 or 115, wherein the detection agent has specificity to the polypeptide.

117. The method of any one of claims 114-116, wherein the detection agent comprises a signal.

118. The method of claim 117, wherein the signal is a detectable label.

119. The method of claim 118, wherein the detectable label is a fluorescent label.

120. The method of any one of claims 75-119, wherein the first APC comprises a nucleic acid molecule encoding the first antigen.

121. The method of claim 114, further comprising sequencing the nucleic acid molecule or a derivative thereof to identify the first antigen.

122. The method of any one of claims 75-121, wherein the plurality of TCRs comprises at least about 2 times more cells than the plurality of APCs.

123. A method for labeling an antigen-presenting cell (APC) comprising an antigen as being recognized by a T-cell receptor (TCR), comprising: contacting an APC comprising an antigen complexed with a major histocompatibility complex (WIC) molecule recognized by a TCR with a TCR-expressing cell comprising the TCR, wherein the TCR-expressing cell secretes a polypeptide upon binding the APC, wherein the polypeptide binds to a catch agent associated with the APC, thereby labeling the APC with the polypeptide, and wherein the catch agent is (i) a soluble catch agent having specificity to both a cell surface protein of the APC and the polypeptide, or (ii) an exogenously expressed protein having specificity to the polypeptide.

124. The method of claim 123, wherein the exogenously expressed protein is a membrane-bound protein.

125. A method of identifying an additional TCR that recognizes an antigen identified by the method of any one of claims 1-124, comprising contacting a plurality of cells, each expressing a different TCR, with the antigen.

126. A composition comprising an antigen identified by the method of any one of claims 1-124.

127. The composition of claim 126, wherein the composition is a pharmaceutical composition comprising a pharmaceutically acceptable carrier.

128. A compartment comprising:
a TCR-expressing cell comprising a T-cell receptor (TCR); and
an antigen-presenting cell (APC) associated with a catch agent, wherein the catch agent is capable of binding to a polypeptide secreted by the TCR-expressing cell, and wherein the catch agent is (i) a soluble catch agent having specificity to both a cell surface protein of the APC and the polypeptide, or (ii) an exogenously expressed protein having specificity to the polypeptide.

129. The compartment of claim 128, wherein the exogenously expressed protein is a membrane-bound protein.

130. The compartment of claim 129, wherein the membrane-bound protein is a receptor.

131. The compartment of claim 128 or 129, wherein the soluble catch agent is (i) a molecule expressed and/or secreted by the APC or (ii) a molecule supplied within the compartment.

132. The compartment of any one of claims 128-131, wherein the soluble catch agent has specificity to a cell surface protein of the APC and the polypeptide.

133. The compartment of any one of claims 128-132, wherein the soluble catch agent binds to both a cell surface protein of the APC and the polypeptide.

134. The compartment of claim 133, wherein the cell surface protein is an endogenous protein or an exogenous protein of the APC.

135. The compartment of any one of claims 128-134, wherein the soluble catch agent is an antibody or a fragment thereof.

136. The compartment of claim 135, wherein the antibody is a bispecific antibody (BsAb).

137. The compartment of any one of claims 128-136, wherein the compartment comprises a plurality of compartments, each compartment of the plurality of compartments comprising a TCR-expressing cell and an APC.

138. The compartment of any one of claims 128-136, wherein the compartment comprises a single APC.

139. The compartment of any one of claims 128-138, further comprising a plurality of polymerizable or gellable polymers and/or monomers.

140. The compartment of claim 139, wherein the plurality of polymerizable or gellable polymers and/or monomers are polymerized or gelled.

141. A compartment comprising:
a TCR-expressing cell comprising a T-cell receptor (TCR); and
an antigen-presenting cell (APC) associated with a solid support, which solid support is further associated with a catch agent that is capable of binding to a polypeptide secreted by the TCR-expressing cell.

142. The compartment of claim 141, wherein the solid support is modified with an antibody or fragment thereof, which antibody binds to a cell surface protein of the APC.

143. The compartment of claim 141 or 142, wherein the catch agent is associated with the solid support via a crosslinker or an affinity binding pair.

144. The compartment of any one of claims 141-143, wherein the solid support is a bead or a hydrogel particle.


SEQUENCE LISTING (7 sequences)

SEQ ID NO: 1 - protein, 21 aa, Artificial Sequence
  Description of Artificial Sequence Synthetic peptide
MWWRLWWLLLLLLLLWPMVWA

SEQ ID NO: 2 - protein, 20 aa, Artificial Sequence
  Description of Artificial Sequence Synthetic peptide
METDTLLLWVLLLWVPGSTG

SEQ ID NO: 3 - protein, 22 aa, Artificial Sequence
  Description of Artificial Sequence Synthetic peptide
MDMRVPAQLLGLLLLWLRGARC

SEQ ID NO: 4 - protein, 16 aa, Artificial Sequence
  Description of Artificial Sequence Synthetic peptide
MPLLLLLPLLWAGALA

SEQ ID NO: 5 - protein, 23 aa, Artificial Sequence
  Description of Artificial Sequence Synthetic peptide
MDAMKRGLCCVLLLCGAVFVSPS

SEQ ID NO: 6 - protein, 16 aa, Artificial Sequence
  Description of Artificial Sequence Synthetic peptide
MLLLLLLLLLLALALA

SEQ ID NO: 7 - protein, 17 aa, Artificial Sequence
  Description of Artificial Sequence Synthetic peptide
MLLLLLLLGLRLQLSLG